SEQUENCE LISTING (14 sequences)

SEQ ID NO: 1 - protein, 63 aa, Homo sapiens
GLTYGSPSEGFTWSDGSPVSYENWAYGEPNNYQNVEYCGELKGDPTMSWNDINCEHLNNWICQ

SEQ ID NO: 2 - protein, 13 aa, Homo sapiens
FTWSDGSPVSYEN

SEQ ID NO: 3 - protein, 13 aa, Homo sapiens
YENWAYGEPNNYQ

SEQ ID NO: 4 - protein, 7 aa, Homo sapiens
VSYENWA

SEQ ID NO: 5 - protein, 13 aa, Homo sapiens
GLTYGSPSEGFTW

SEQ ID NO: 6 - protein, 13 aa, Homo sapiens
NYQNVEYCGELKG

SEQ ID NO: 7 - protein, 13 aa, Homo sapiens
LKGDPTMSWNDIN

SEQ ID NO: 8 - protein, 13 aa, Homo sapiens
DINCEHLNNWICQ

SEQ ID NO: 9 - protein, 13 aa, Homo sapiens
DCTSAGRSDGWLW

SEQ ID NO: 10 - protein, 13 aa, Homo sapiens
LVGNIEQNEHSVI

SEQ ID NO: 11 - protein, 471 aa, Streptococcus pneumoniae
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWNELSYDHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 12 - protein, 1456 aa, Homo sapiens [sequence omitted]

SEQ ID NO: 13 - protein, 529 aa, Listeria monocytogenes
MKKIMLVFITLILISLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEINYDPEGNEIVQHKNWSENNKSKLAHFTSSIYLPGNARNINVYAKECTGLAWEWWRTVIDDRNLPLVKNRNISIWGTTLYPKYSNSVDNPIE

SEQ ID NO: 14 - protein, 571 aa, Streptococcus pyogenes
MSNKKTFKKYSRVAGLLTAALIIGNLVTANAESNKQNTASTETTTTSEQPKPESSELTIEKAGQKMDDMLNSNDMIKLAPKEMPLESAEKEEKKSEDKKKSEEDHTEEINDKIYSLNYNELEVLAKNGETIENFVPKEGVKKADKFIVIERKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFKDLQRKGVSNEAPPLFVSNVAYGRTVFVKLETSSKSNDVEAAFSAALKGTDVKTNGKYSDILENSSFTAVVLGGDAAEHNKVVTKDFDVIRNVIKDNATFSRKNPAYPISYTSVFLKNNKIAGVNNRTEYVETTSTEYTSGKINLSHQGAYVAQYEILWDEINYDDKGKEVITKRRWDNNWYSKTSPFSTVIPLGANSRNIRIMARECTGLAWEWWRKVIDERDVKLSKEINVNISGSTLSPYGSITYK

(19) United States
(12) Patent Application Publication
(10) Pub. No.: US 2023/0365647 A1
(43) Pub. Date: Nov. 16, 2023

(54) MANNOSE RECEPTOR-DERIVED PEPTIDES FOR NEUTRALIZING PORE-FORMING TOXINS FOR THERAPEUTIC USES

(71) Applicant: ZALVAC AB, Stockholm (SE)

(72) Inventors: Birgitta HENRIQUES NORMARK, Stockholm (SE); Karthik SUBRAMANIAM, Thiruvananthapuram (IN); Georgios SOTIRIOU, Akersberga (SE)

(21) Appl. No.: 18/013,679

(22) PCT Filed: Jun. 29, 2021
(86) PCT No.: PCT/EP2021/067923

§ 371 Date: Dec. 29, 2022

(30) Foreign Application Priority Data
Jun. 30, 2020 (SE) 2050805-7

(51) Int. Cl. C07K 14/705; A61P 31/04; A61K 9/00

(52) U.S. Cl. CPC C07K 14/705; A61P 31/04; A61K 9/0043

(57) ABSTRACT

A polypeptide or peptidomimetic comprising a sequence of at least 7 residues differing by residue substitutions, deletions or insertions numbering 0-2 compared to the sequence: GLTYGSPSEGFTWSDGSPVSYENWAYGEPNNYQNVEYCGELKGDPT-MSWNDINCEHLNNWICQ (SEQ ID NO: 1), for use as a medicament, in particular in pneumococcal disease.

### TECHNICAL FIELD The present invention relates to therapeutic compounds for use in the treatment of disease caused by micro-organisms expressing pore-forming toxins, such as pneumococcal disease.

### BACKGROUND TO THE INVENTION

Bacterial infections are leading causes of mortality and morbidity worldwide, and the emergence of resistance to many antibiotics is a major threat to society. A common characteristic of many pathogenic bacteria, including some that have evolved drug resistance, is that they employ pore-forming toxins (PFTs) as virulence factors. PFTs constitute more than one-third of all cytotoxic toxins, making them the largest category of bacterial virulence factors. Cholesterol-dependent cytolysins (CDCs) are a subclass of β-PFTs that bind to the cholesterol on eukaryotic cells, and form barrel-shaped pores to mediate cytolysis. PFTs promote bacterial virulence in many ways such as (i) induction of epithelial barrier dysfunction, (ii) lysis of phagocytic immune cells, and (iii) aiding bacterial invasion of host cells and intracellular survival. Prominent examples of bacterial CDCs include pneumolysin (PLY) of S. pneumoniae, streptolysin O (SLO) of S. pyogenes, and listeriolysin O (LLO) of L. monocytogenes.

The structure of CDCs is conserved, consisting of four domains, and domain 4 is known to bind to the eukaryotic cell membrane (R. K. Tweten, Cholesterol-dependent cytolysins, a family of versatile pore-forming toxins. Infection and immunity 73, 6199-6209 (2005)). Specifically, the highly conserved tryptophan rich-undecapeptide loop in domain 4 has been shown to bind cholesterol on eukaryotic membranes (K. van Pee et al., CryoEM structures of membrane pore and prepore complex reveal cytolytic mechanism of Pneumolysin. eLife 6, (2017)). The binding triggers oligomerization of membrane bound monomeric toxins into pre-pore structure. Conformational change triggers two α-helices in domain 3 to unfold into β-hairpins which then insert into the membrane to form 250-300 Å pores. Due to their ubiquitous expression in bacterial pathogens, CDCs are attractive targets for development of novel broadly applicable antimicrobial therapeutics. The application of antibiotics to treat bacteremic patients is known to cause release of CDCs from lysed bacteria, and hence adjunctive therapies to ameliorate the tissue damage caused by the released toxins are needed.

The inventors recently showed that at sublytic doses, PLY binds to the mannose receptor C type 1 (MRC-1) on dendritic cells (DCs) and lung alveolar macrophages, resulting in an anti-inflammatory response and enhanced intracellular survival of pneumococci (K. Subramanian et al., Pneumolysin binds to the mannose receptor C type 1 (MRC-1) leading to anti-inflammatory responses and enhanced pneumococcal survival. Nat Microbiol 4, 62-70 (2019)). Soluble recombinant polypeptides comprising at least one carbohydrate recognition domain of MRC-1 (that generally contains at least 150 amino acids), and their medical uses have been disclosed in WO92/07579.

Previously, antibodies targeting PLY, cholesterol loaded decoy liposomes, and phytosterols resembling cholesterol, have been found to protect mice against S. pneumoniae infection (H. Li et al., beta-sitosterol interacts with pneumolysin to prevent Streptococcus pneumoniae infection. Scientific reports 5, 17668 (2015); D. M. Musher, H. M. Phan, R. E. Baughn, Protection against bacteremic pneumococcal infection by antibody to pneumolysin. The Journal of infectious diseases 183, 827-830 (2001); B. D. Henry et al., Engineered liposomes sequester bacterial exotoxins and protect from severe invasive infections in mice. Nature biotechnology 33, 81-88 (2015)). However, these strategies can cause undesired immune activation.

Thus, an object of the present invention is the provision of alternative and/or improved methods and/or means for neutralizing CDCs and/or to reduce inflammation and/or to treat diseases caused by CDC-expressing bacteria, such as pneumococcal disease.

### DEFINITIONS

The term sequence identity expressed in percentage is defined as the value determined by comparing two optimally aligned sequences over a comparison window, wherein a portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. Unless indicated otherwise, the comparison window is the entire length of the sequence being referred to. In this context, optimal alignment is the alignment produced by the BLASTP algorithm as implemented online by the US National Center for Biotechnology Information (see The NCBI Handbook, 2^{nd} edition [https://www.ncbi.nlm.nih.gov/books/NBK143764/]), with the following input parameters: Word length=3, Matrix=BLOSUM62, Gap cost=11, Gap extension cost=1.

The term treatment in the present context refers to treatments resulting in a beneficial effect on a subject afflicted with the condition to be treated, including any degree of alleviation, including minor alleviation, substantial alleviation, major alleviation as well as cure. Preferably, the degree of alleviation is at least a minor alleviation.

The term prevention in the present context refers to preventive measures resulting in any degree of reduction in the likelihood of developing the condition to be prevented, including a minor, substantial or major reduction in likelihood of developing the condition as well as total prevention. Preferably, the degree of likelihood reduction is at least a minor reduction.

Pneumolysin (termed Ply or PLY herein) is a 53 kDa cholesterol dependent cytolysin expressed by Streptococcus pneumoniae. It is one of the major virulence factors of this bacterium. It forms pores in all eukaryotic cells that have cholesterol in their membranes. The formation of pores by PLY frequently results in host cell death as membrane integrity is destroyed. PLY plays a central role in protecting the pneumococcus from complement attack and aiding its spread to other tissues/organs. PLY is able to activate the classical complement pathway, even in the absence of PLY specific antibody (Mitchell & Dalziel. Subcell Biochem. 2014; 80:145-60). A reference sequence from strain TIGR4 is presented in SEQ ID NO: 11.

Streptolysin O (termed Slo or SLO herein) is the hemolytic toxin produced by most strains of Group A Streptococci (Streptococcus pyogenes). SLO is oxygen labile and belongs to the family of cholesterol binding toxins such as pneumolysin. A reference sequence from Streptococcus pyogenes is presented in SEQ ID NO: 14.

Listeriolysin O (termed llo or LLO herein) is a hemolysin produced by the bacterium Listeria monocytogenes that causes listeriosis. Like PLY and SLO, LLO is a cholesterol dependent pore-forming toxin. The unique feature of LLO is that its cytolytic activity is maximal at acidic pH. A reference sequence from Listeria monocytogenes is presented in SEQ ID NO: 13.

Mannose receptor C type 1 (termed MRC-1 herein) is a C type 1 transmembrane endocytic receptor that is primarily expressed on the surface of dendritic cells, tissue macrophages such as alveolar macrophages in the lungs. The structure of MRC-1 consists of a N-terminal cysteine rich domain, fibronectin type II repeat domain, 8 C type lectin domains and a short intracellular tail. Known biological functions of MRC-1 include binding to terminal mannose, N-acetylglucosamine and fucose residues on proteins found on the surface of microorganisms as well as clearance of glycoprotein hormones from the blood. A reference sequence from Homo sapiens is presented in SEQ ID NO: 12.

### BRIEF DESCRIPTION OF THE FIGURES

FIG. 1. MRC-1 co-localizes with the bacterial CDCs, PLY, LLO and SLO in human dendritic cells. (A-C). Human DCs were incubated with a non-cytolytic dose (0.2 μg/ml) of purified PLY, LLO or SLO for 45 min. Immunofluorescence staining shows that PLY, LLO and SLO (green) co-localize with MRC-1 (red) in DCs and EEA-1 (early endosomes). All scale bars, 5 μm. Images are representative of three independent experiments.

FIG. 2. The CTLD4 domain of MRC-1 interacts with the cholesterol binding loop of bacterial CDCs. Computational docking of (A) PLY, (B) LLO and (C) SLO (in green) with the CTLD4 domain of MRC-1 (in red) was performed using the ClusPro 2.0 docking server. Modeling based on least energy configurations indicate that the unstructured loop of MRC-1 docks to the conserved cholesterol binding loop (in yellow) of PLY, LLO and SLO. The amino acid residues involved in polar interactions are zoomed in below. (D-E) 3D view of the CTLD4 domain of MRC-1 showing the surface location of the peptides P2 and P3 (in pink). Acidic and basic amino acid residues are shown in red and blue respectively. Indicated in green is the calcium binding site.

FIG. 3. MRC-1 peptides bind to bacterial CDCs and inhibit their induction of hemolysis and cytolysis of macrophages. (A) ELISA showing the dose-dependent binding of plate-bound MRC-1 peptides P2, P3 and the control peptides CP1 and CP2 to PLY (0-0.5 μM). BSA was used as negative control to show the binding specificity. Data are mean±s.e.m. of two independent experiments, each containing three replicates per condition. (B) Hemolysis assay (n=4) of 1 μg/ml purified PLY in the presence of increasing concentrations of MRC-1 peptides, P2, P3 and control peptide CP2(1-1000 μM). ** denotes P<0.005 by one-way ANOVA with Dunnett's post test. (C) LDH cytotoxicity assay in human THP-1 macrophages stimulated with purified PLY, LLO or SLO (0.5 μg/ml) in the presence or absence of 100 μM peptides P2, P3 or control peptide CP2 for 18 h. Cholesterol (100 μM) was used as positive control to inhibit hemolysis. Data are mean±s.e.m from 4 independent experiments. **** denotes P<0.0001 by two-way ANOVA with Bonferroni post test. n.s. denotes not significant. (D) Binding of FITC-labelled peptides P2 and CP2 to wild-type pneumococci, TIGR4 (T4) and isogenic PLY mutant (T4Δply) was visualized by fluorescence microscopy. Scale bars, 10 μm. Images are representative of three independent experiments. (E) The hemolytic activity of wild-type pneumococci, TIGR4 (T4) and PLY mutant, T4Δply in the presence of 100 μM peptide P2 and CP2. Data are the mean±s.e.m. of three independent experiments. *** denotes P<0.005 by one-way ANOVA with Dunnett's post test. n.s. denotes not significant.

FIG. 4. PLY-mediated bacterial invasion of the lung epithelium and intracellular bacterial survival are inhibited by MRC-1 peptides. (A) IL-8 released by human THP-1 macrophages stimulated with purified PLY, LLO or SLO (0.5 μg/ml) in the presence or absence of 100 μM peptides P2, P3 or control peptide CP2 for 18 h. Cholesterol (100 μM) was used as positive control to inhibit hemolysis. Data are mean±s.e.m from three independent experiments. **** denotes P<0.0001 by two-way ANOVA with Bonferroni post test. n.s. denotes not significant. (B) Schematic showing the cellular architecture of the 3D lung epithelial model. (C) 3D volume images of the GFP-lung epithelial models at 1 h and 3 h post stimulation with 50 μg PLY in the presence or absence of 100 μM peptide P2 or the control peptide CP2. Images are representative of two independent experiments with n=3 models/condition. (D) Invasion of wild-type pneumococci T4 (TIGR4) or its isogenic PLY mutant T4Δply into the lung epithelial models (n=3/condition) in the presence or absence of 100 μM peptide P2 or the control peptide CP2 at 2h post infection was measured using CFU viability assay following gentamicin killing of extracellular bacteria. Anti-PLY was used as control to test the effect of blocking PLY. Data in d and e are mean±s.e.m. of n=3 models/condition from two independent experiments. ** denotes P<0.005 by one-way ANOVA with Dunnett's post test. n.s. denotes not significant. (E) Human DCs were infected with type 4 and type 2 pneumococci, T4 and D39 respectively, at MOI of 10 in the presence or absence of 100 μM peptides, P2 or CP2, and intracellular bacteria were counted at 3 h post infection following gentamicin killing of extracellular bacteria. Cytochalasin D (0.5 mM) was used as negative control to inhibit phagocytosis. Anti-PLY was used as control to test the effect of blocking PLY. Data are mean±s.e.m. of three independent experiments. **** denotes P<0.0001 by two-way ANOVA with Bonferroni post test. n.s. denotes not significant. (F) DCs were infected with the unencapsulated pneumococci, T4R, or (G) its isogenic PLY mutant, T4RΔpl,y in the presence of 100 μM peptides, P2 or CP2 at MOI of 10 for 2 h. Immunofluorescence microscopy images show that in DCs treated with peptide P2 (but not the control peptide CP2), intracellular T4R (green) do not co-localize with MRC-1 (red), but with the autophagy protein LC3B (cyan). Peptide P2 had no effect on the co-localization of T4RΔply (green) which always co-localized with LC3B (pink), but not with MRC-1(red). Images are representative of three independent experiments. Scale bars, 10 μm.

FIG. 5. Treatment with peptide P2 reduces development of pneumococcal disease in vivo. (A) Survival percentage of 3-4 dpf zebrafish embryos (n≥156) upon infection with S. pneumoniae T4 alone or its isogenic PLY mutant, T4Δply. Injection with E3 growth medium served as mock control. (B) Zebrafish survival percentage upon infection with T4 alone or together with peptide P2 or CP2 or P2-conjugated CaP NPs (P2-NPs). *** denotes P<0.0005 and **** denotes P<0.0001 by Mantel Cox test. (C) Survival of mice (n=10) upon intranasal infection with 2×10⁶ CFU of S. pneumoniae T4 together with peptide P2 or CP2 or P2-NPs over 3 days post infection. Infected mice were checked twice daily in the morning at 9 am (early check) and in evening at 7 pm (late check) for clinical symptoms. Unloaded NPs and the isogenic PLY mutant strain (T4Δply) served as negative controls. * denotes P<0.05 and ** denotes P<0.005 by Mantel Cox test. (D) Bacterial load and levels of pro-inflammatory cytokines (E) TNF-α and (F) IL-12 in the lungs of infected mice (n=10) were measured post sacrifice. * denotes P<0.05, ** denotes P<0.005 and **** denotes P<0.0001 by one-way ANOVA with Bonferroni's post test. n.s. denotes not significant.

FIG. 6. Model showing mechanisms by which MRC-1 peptides reduce pneumococcal disease. (Left) S. pneumoniae produces the cholesterol dependent cytolysin (CDC), pneumolysin (PLY), that induces pore-formation and lysis of host macrophages (Mfs). At non-cytolytic doses, PLY:MRC-1 interaction promotes bacterial invasion and intracellular survival in dendritic cells (DCs) and macrophages. (Right) The inventors developed MRC-1 peptides conjugated nanoparticles (NPs) as treatment to reduce pneumococcal infection. The peptides bind and inhibit PLY-induced lysis of host cells. Further, the peptides inhibit bacterial internalization into DCs via MRC-1 and promote autophagy killing of intracellular bacteria. Mice treated with peptide P2-NPs show higher survival upon pneumococcal infection as well as reduced lung bacterial load and inflammation.

FIG. 7 The toxoids PLY (W433F) and LLO (W489F) do not co-localize with MRC-1. Human DCs were incubated with a non-cytolytic dose (0.2 μg/ml) of purified toxoid derivatives, (A) PLY(W433F) and (B) LLO(W489F) for 45 min. Immunofluorescence staining shows that both PLY(W433F) and LLO(W489F) (green) show weak binding to DCs and do not co-localize with MRC-1 (red) in DCs. All scale bars, 5 μm. Images are representative of three independent experiments.

FIG. 8 Location and activity of MRC-1 peptides against PLY and LLO. (A) Domain architecture and location of peptides (P1-P6, CP1 and CP2) on the MRC-1 protein. P1-P6 are from the CTLD4 domain which binds to the membrane binding loop of CDCs while the control peptides, CP1 and CP2, are from regions that do not bind CDCs. (B) Red blood cell hemolysis assay showing the residual cell pellet after hemolysis by purified PLY and LLO (1 μg/ml) in the presence of 100 μM of MRC-1 peptides. Complete hemolysis indicated by absence of red pellet was achieved by PLY and LLO alone as well as the control peptides, CP1 and CP2, while peptides P1-P6 conferred protection to various extents. Cholesterol was used as positive control to block hemolysis. Images are representative of three experiments. (C) Quantification of hemolysis induced by PLY(1 μg/ml) in the presence of 100 μ.M of MRC-1 peptides, P1-P6, and control peptides, CP1 and CP2. BSA was used as negative control to show specificity while cholesterol was used as positive control to block hemolysis. Data are mean±s.e.m from two experiments with triplicates. *** denotes P<0.001 by one-way ANOVA with Dunnett's posttest. n.s. denotes not significant.

FIG. 9 Dose-dependent binding of peptides to LLO and SLO and inhibition of hemolysis and pro-inflammatory cytokine responses. ELISA showing the dose-dependent binding of plate bound MRC-1 peptides P2, P3 and the control peptides CP1 and CP2 to (A) purified LLO and (B) SLO (0-0.5 μM). BSA was used as negative control to show the binding specificity. Data are mean±s.e.m from two independent experiments with triplicates. (C) Hemolysis assay of 1 μg/ml purified LLO and (D) SLO in the presence of increasing concentrations of MRC-1 peptides, P2, P3 and control peptide CP2(1-1000 μM). Data are mean±s.e.m from 4 independent experiments. ** denotes P<0.005 by one-way ANOVA with Dunnett's post test. (E) Hemolytic activity of S. pyogenes type M1T1 and (F) L. monocytogenes in the presence of 100 μM peptide P2 and CP2. The isogenic SLO and LLO mutant strains were used as negative controls. Data in e, f are mean±s.e.m from three independent experiments. *** denotes P<0.005 by one-way ANOVA with Dunnett's post test. n.s. denotes not significant.

FIG. 10 The MRC-1 peptide P2 inhibits hemolysis and intracellular survival of bacteria in human DCs. (A) IL-12 and (B) TNF-α release by human THP-1 macrophages stimulated with purified PLY, LLO or SLO (0.5 μg/ml) in the presence or absence of 100 μM peptides P2, P3 or control peptide CP2 for 18 h. Cholesterol (100 μM) was used as positive control to inhibit hemolysis. Data are mean±s.e.m from three independent experiments. *** denotes P<0.001 and **** denotes P<0.0001 by two-way ANOVA with Bonferroni post test. n.s. denotes not significant. (C) The loss of the GFP signal intensity owing to cell death at 3 h post stimulation of GFP-expressing 3D lung epithelial models (n=3) was quantified relative to 1 h time point. * indicates * denotes P<0.05 by paired t test. (D) Cytotoxicity of the lung epithelial model stimulated with 50 μg PLY in the presence or absence of 100 μM peptide P2 or the control peptide CP2 at 18 h. *** denotes P<0.001 by one-way ANOVA with Dunnett's post test. (E) TNF-α and (F) IL-8 release by the lung epithelial model stimulated with 50 μg PLY in the presence or absence of 100 μM peptide P2 or the control peptide CP2 at 18 h. ** denotes P<0.005; *** denotes P<0.001 by one-way ANOVA with Dunnett's post test. Data in c-f are mean±s.e.m from two independent experiments with n=3 model/condition. (G) Immunofluorescence microscopy images showing intracellular pneumococci of type 4 or type 2 in infected human DCs treated or not with 100 μM peptide P2 or the control peptide CP2. Anti-PLY antibody was used as control to block PLY. DCs infected with D39 or T4 alone possessed intracellular bacteria (green) that co-localized with MRC-1 (red). Arrows indicate MRC-1 co-localized intracellular bacteria (yellow). DCs treated with peptide P2, but not CP2, were devoid of intracellular bacteria. Images are representative of two independent experiments.

FIG. 11 Intracellular bacterial localization in infected DCs upon treatment with MRC-1 peptides. Quantification of percentage of intracellular (A) S. pneumoniae and (B) S. pyogenes (n=50) in infected DCs that co-localize with MRC-1 and LC3B. Data are mean±s.e.m from two independent experiments. **** denotes P<0.0001 by two-way ANOVA with Bonferroni post test. n.s. denotes not significant. (C) DCs were infected with S. pyogenes (S.py) type M1T1 (left panel) and the isogenic SLO mutant, S. pyΔslo (right panel) in the presence of 100 μM peptides, P2 or CP2 at MOI of 10 for 2 h. Immunofluorescence microscopy images show that in infected DCs treated with peptide P2 (but not the control peptide CP2), intracellular streptococci (green) do not colocalize with MRC-1 (red), but with the autophagy protein, LC3B (cyan). Peptide P2 had no effect on colocalization of S. pyΔslo (green) which always colocalized with LC3B (pink), but not with MRC-1(red). Scale bars, 10 μm.

FIG. 12 Characterization of peptide-loaded CaP NPs and testing of lung delivery and toxicity in vivo. (A) TEM image of calcium phosphate (CaP) nanoparticles (NPs), exhibiting the characteristic fractal-like agglomerate structure. Scale bar, 50 nm. (B) Size distribution of CaP NPs before and after loading with MRC-1 peptide in pure H₂O (solid lines) and PBS (broken lines) was determined by Dynamic light scattering (particle concentration 100 μg/ml). (C) Loading capacity of MRC-1 peptides, P2 and CP2 (concentration 100 μg/ml), as a function of NP concentration after overnight co-incubation at room temperature. The loaded amount of MRC-1 peptide is decreased when the particle concentration is increased (constant input peptide concentration 100 μg/ml), probably because of increased agglomeration at higher particle concentrations resulting in a lower available surface area for bioconjugation. (D) Dose-dependent inhibition of S. pneumoniae T4 induced hemolysis by MRC-1 peptide loaded CaP NPs (peptide content 0-0.5 μg, CaP NPs 0.25-1.25 mg/ml). NPs loaded with the control peptide, CP2, and unloaded NPs served as negative controls. * denotes P<0.05 by two-way ANOVA with Dunnett's post-test. (E) IVIS imaging showing the lung distribution of NPs loaded with Cy7 tagged peptide P2 (5 μg peptide; 25 μg CaP NPs/mouse) was measured at 1 h and 24 h post intranasal instillation of NPs in mice. Unloaded NPs and Cy7 dye alone served as negative controls. (F) Hematoxylin and eosin (H&E) staining of mouse lungs at 24 h post intranasal administration of NPs alone (25 μg CaP NPs/mouse) or P2-NPs (5 μg peptide; 25 μg CaP NPs/mouse) or P2 alone (5 μg/mouse). Peptide P2 or P2-NPs were not toxic as revealed by the lung histology analysis which showed that the mice had a normal lung morphology with intact alveolar space (Al) (magnified in the inset) and absence of inflammatory cells. Al-alveolar space; Br-bronchiole; Bv-blood vessel. Scale bars, 50 μm.

### SUMMARY OF THE INVENTION

The present invention relates to the following items. The subject matter disclosed in the items below should be regarded disclosed in the same manner as if the subject matter were disclosed in patent claims.
1. A polypeptide or peptidomimetic comprising:
   a sequence of at least 7 residues differing by residue substitutions, deletions or insertions numbering 0-2 compared to the sequence:

(SEQ ID NO: 1)
GLTYGSPSEGFTWSDGSPVSYENWAYGEPNNYQNVEYCGELKGDPTMSW
NDINCEHLNNWICQ,

for use as a medicament.
2. The polypeptide or peptidomimetic according to item 1, for use in the treatment or prevention of a bacterial infection by a pathogen expressing a cholesterol-dependent cytolysin (CDC).
3. The polypeptide or peptidomimetic according to any of the preceding items, for use in neutralizing bacterial CDCs in a subject.
4. The polypeptide or peptidomimetic according to any of the preceding items, for use as a medicament in the treatment or prevention of pneumococcal disease.
5. The polypeptide or peptidomimetic according to any of the preceding items, for use as a medicament in the treatment or prevention of pneumococcal disease selected from pneumococcal sinusitis, pneumococcal otitis, pneumococcal pneumonia and invasive pneumococcal disease including but not limited to pneumococcal sepsis and pneumococcal meningitis, preferably invasive pneumococcal disease.
6. The polypeptide or peptidomimetic according to any of the preceding items, for use in the treatment or prevention of secondary bacterial pneumonia following a virus infection.
7. The polypeptide or peptidomimetic according to any of items 1-3, for use in the treatment or prevention of listeriosis.
8. The polypeptide or peptidomimetic according to any of items 1-3, for use in the treatment or prevention of necrotizing fasciitis.
9. The polypeptide or peptidomimetic for use according to any of the preceding items, wherein the use involves intranasal administration of the polypeptide or peptidomimetic to a subject in need thereof.
10. The polypeptide or peptidomimetic for use according to any of the preceding items, wherein the use further involves administration of antibiotics.
11. The polypeptide or peptidomimetic for use according to any of the preceding items, wherein the sequence comprises YEN residues aligning with the bolded sequence at positions 21-23 of SEQ ID NO: 1.
12. The polypeptide or peptidomimetic for use according to any of the preceding items, wherein the sequence comprises a T residue aligning with the position 12 of SEQ ID NO: 1.
13. The polypeptide or peptidomimetic for use according to any of the preceding items, wherein the sequence comprises a S residue aligning with the position 14 of SEQ ID NO: 1.
14. The polypeptide or peptidomimetic for use according to any of the preceding items, wherein the polypeptide or peptidomimetic comprises the sequence VSYENWA (SEQ ID NO: 4).
15. The polypeptide or peptidomimetic for use according to any of the preceding items, wherein the polypeptide or peptidomimetic comprises the sequence FTWSDGSPVSYEN (SEQ ID NO: 2), or a subsequence thereof being at least 5 residues long, preferably at least 6, more preferably at least 7, yet more preferably at least 8, still more preferably at least 9, even more preferably at least 10, further still preferably at least 11, most preferably at least 12 residues long.
16. The polypeptide or peptidomimetic for use according to any of the preceding items, wherein the polypeptide or peptidomimetic comprises YENWAYGEPNNYQ (SEQ ID NO: 3), or a subsequence thereof being at least 5 residues long, preferably at least 6, more preferably at least 7, yet more preferably at least 8, still more preferably at least 9, even more preferably at least 10, further still preferably at least 11, most preferably at least 12 residues long.
17. The polypeptide or peptidomimetic for use according to any of the preceding items, wherein the polypeptide or peptidomimetic comprises a consecutive sequence of at least 8, preferably at least 9, more preferably at least 10, yet more preferably at least 11, still more preferably at least 12, most preferably at least 13 residues differing by residue substitutions, deletions or insertions numbering 0-2 compared to the sequence SEQ ID NO:1.
18. The polypeptide or peptidomimetic for use according to any of the preceding items, wherein the polypeptide or peptidomimetic comprises a consecutive sequence of no more than 60 residues having more than 80% sequence identity to SEQ ID NO:1, preferably no more than 50, more preferably no more than 40, yet more preferably no more than 30, most preferably no more than 20.
19. The polypeptide or peptidomimetic for use according to any of the preceding items, wherein the polypeptide or peptidomimetic comprises no more than 100 residues in total, preferably no more than 60 residues, preferably no more than 50, more preferably no more than 40, yet more preferably no more than 30, most preferably no more than 20.
20. The polypeptide or peptidomimetic for use according to any of the preceding items, wherein the residue substitutions, deletions or insertions number 2 in total.
21. The polypeptide or peptidomimetic for use according to any of the preceding items, wherein the residue substitutions, deletions or insertions number 1 in total.
22. The polypeptide or peptidomimetic for use according to any of the preceding items, wherein the residue substitutions, deletions or insertions number 0 in total.
23. The polypeptide or peptidomimetic for use according to any of the preceding items, wherein the deletions or insertions number 0 in total.
24. The polypeptide or peptidomimetic for use according to any of the preceding items, wherein the sequence comprises at least one difference compared to any naturally occurring sequence and/or the polypeptide or peptidomimetic comprises a non-naturally occurring chemical moiety.
25. The polypeptide or peptidomimetic for use according to any of the preceding items, wherein the polypeptide or peptidomimetic is a polypeptide.
26. The polypeptide or peptidomimetic for use according to any of items 1-24, wherein the polypeptide or peptidomimetic is a peptidomimetic.
27. The polypeptide or peptidomimetic for use according to any of the preceding items, having a modified C-terminal, such as an amidated C-terminal.
28. The polypeptide or peptidomimetic for use according to any of the preceding items, having a modified N-terminal, such as an acylated N-terminal.
29. The polypeptide or peptidomimetic for use according to any of the preceding items, having a cyclic backbone.
30. The polypeptide or peptidomimetic for use according to any of the preceding items, comprising one or more non-natural residues.
31. The polypeptide or peptidomimetic for use according to any of the preceding items, comprising one or more D-amino acid residues.
32. The polypeptide or peptidomimetic for use according to any of the preceding items, wherein one or more the P residues is/are hydroxylated.
33. The polypeptide or peptidomimetic for use according to any of the preceding items, comprising one or more non-peptide bonds in the backbone.
34. The polypeptide or peptidomimetic for use according to any of the preceding items, conjugated to a detectable marker, preferably biotin, a fluorescent marker, or a radioactive label.
35. The polypeptide or peptidomimetic for use according to any of the preceding items, conjugated to a chemical moiety increasing residence time in plasma, such a polyethylene glycol, PAS-group, albumin, or the like.
36. The polypeptide or peptidomimetic for use according to any of the preceding items, wherein the polypeptide or peptidomimetic binds to the cholesterol binding loop of 25 bacterial cholesterol-dependent cytolysins, preferably Pneumolysin (PLY), streptolysin (SLO) or listeriolysin (LLO), most preferably PLY.
37. The polypeptide or peptidomimetic for use according to any of the preceding items, wherein the polypeptide or peptidomimetic binds to PLY, SLO or LLO, preferably PLY.
38. The polypeptide or peptidomimetic for use according to any of the preceding items, wherein the polypeptide or peptidomimetic is inhibitory to hemolysis by PLY, SLO or LLO, preferably PLY.
39. The polypeptide or peptidomimetic for use according to any of the preceding items, wherein the polypeptide or peptidomimetic is inhibitory to hemolysis by PLY, SLO or LLO, preferably PLY, with an IC50 of less than 1000 μM, preferably 100 μM, more preferably 10 μM in a hemolysis assay.
40. The polypeptide or peptidomimetic for use according to any of the preceding items, wherein the polypeptide or peptidomimetic is inhibitory to hemolysis by PLY with an IC50 of less than 1000 μM (preferably 100 μM, more preferably 10 μM) in an assay using blood diluted 1:100 in physiological phosphate-buffered saline combined with 1 μg/ml purified PLY at 37 C for 1 h.
41. The polypeptide or peptidomimetic for use according to any of the preceding items, wherein the polypeptide or peptidomimetic competitively interferes with the cholesterol-binding activity of PLY, SLO or LLO, preferably PLY.
42. The polypeptide or peptidomimetic for use according to any of the preceding items, wherein the polypeptide or peptidomimetic is formulated as a composition according to item 43, or any item dependent thereon.
43. A composition, comprising a polypeptide or peptidomimetic comprising:
   a sequence of at least 7 residues differing by residue substitutions, deletions or insertions numbering 0-2 compared to the sequence:

(SEQ ID NO: 1)
GLTYGSPSEGFTWSDGSPVSYENWAYGEPNNYQNVEYCGELKGDPTMSW
NDINCEHLNNWICQ,

wherein the polypeptide or peptidomimetic is immobilized on a carrier.
44. The composition according to item 43, wherein the polypeptide or peptidomimetic is as disclosed in item 11 or any item dependent thereon.
45. The composition according to item 43 or any item dependent thereon, wherein the carrier is a nanoparticle.
46. The composition according to item 43 or any item dependent thereon, wherein the carrier is an inorganic or polymeric nanoparticle.
47. The composition according to item 43 or any item dependent thereon, wherein the carrier is a nanoparticle comprising calcium phosphate, gold, iron oxide, silica, poly(lactic-co-glycolic acid) (PLGA), poly(lactic acid) (PLA) or poly(amino acids) such as poly(γ-glutamic acid) (γ-PGA), poly(ε-lysine), poly(L-arginine), poly(L-histidine), gallium oxide, or gallium phosphate, preferably calcium phosphate.
48. The composition according to item 43 or any item dependent thereon, wherein the carrier contains 10-1000 mg peptide/g carrier, more preferably 50-500 mg peptide/g carrier, even more preferably 50-300 mg/g carrier, yet more preferably 50-200 mg/g carrier, most preferably about 75-150 mg/g carrier.
49. The composition according to item 43 or any item dependent thereon, wherein the carrier is a nanoparticle with size in the range of from 5 to 100 nm, preferably 10-20 nm.
50. The composition according to item 49, wherein the agglomerate size of the nanoparticles in water is from 50 to 1000 nm, preferably 200-700 nm, most preferably 100-500 nm.
51. The composition according to item 43 or any item dependent thereon, wherein the peptide is associated with the carrier through covalent or non-covalent binding, preferably through physical adsorption.
52. A polypeptide or peptidomimetic comprising:
   a sequence of at least 7 residues differing by residue substitutions, deletions or insertions numbering 0-2 compared to the sequence:

(SEQ ID NO: 1)
GLTYGSPSEGFTWSDGSPVSYENWAYGEPNNYQNVEYCGELKGDPTMSW
NDINCEHLNNWICQ.

53. The polypeptide or peptidomimetic according to item 52, wherein the peptide or peptidomimetic is as disclosed in item 11 or any item dependent thereon.

### DETAILED DESCRIPTION

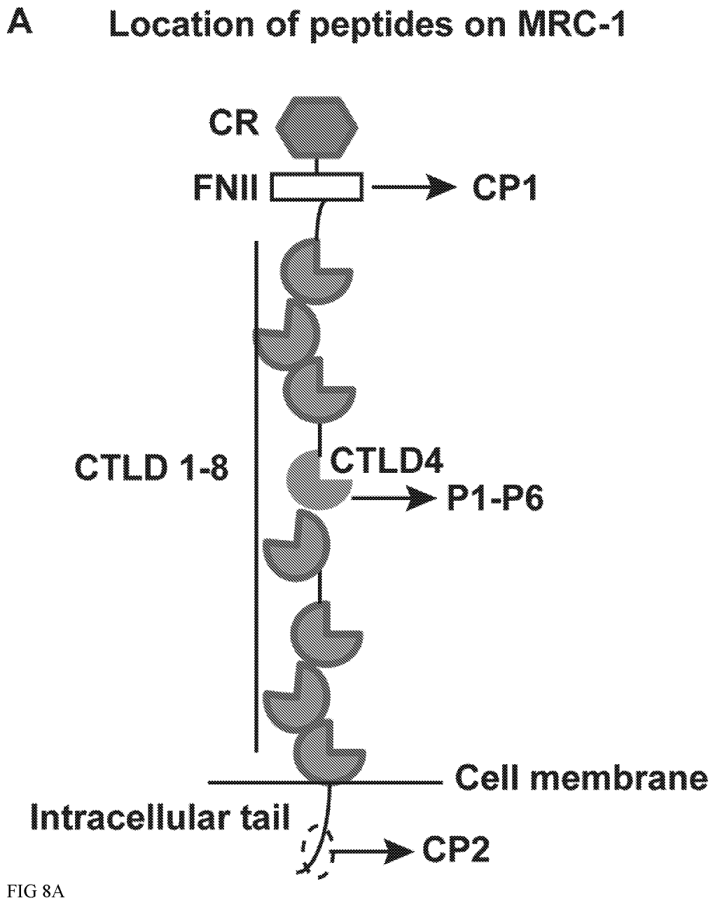

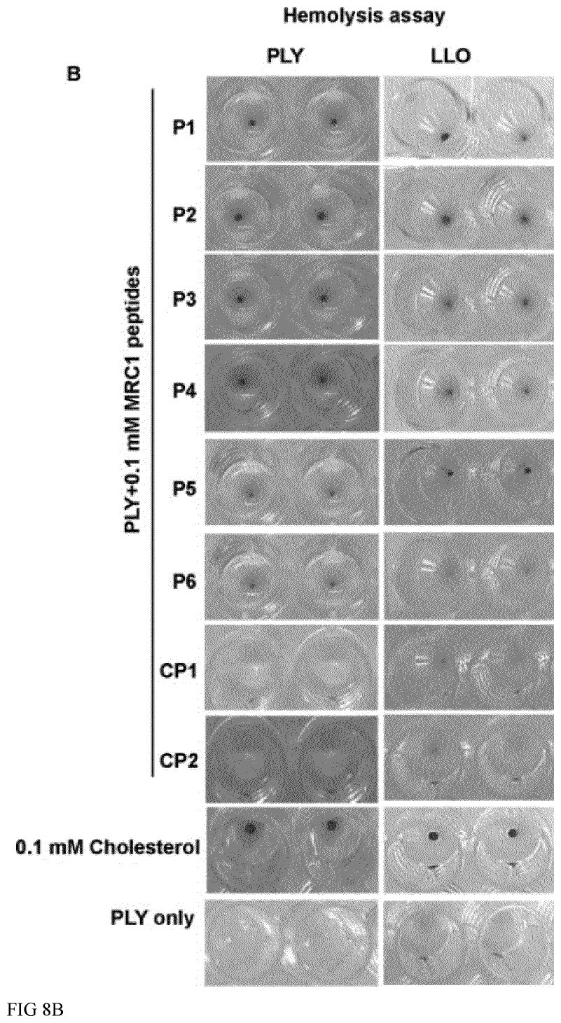

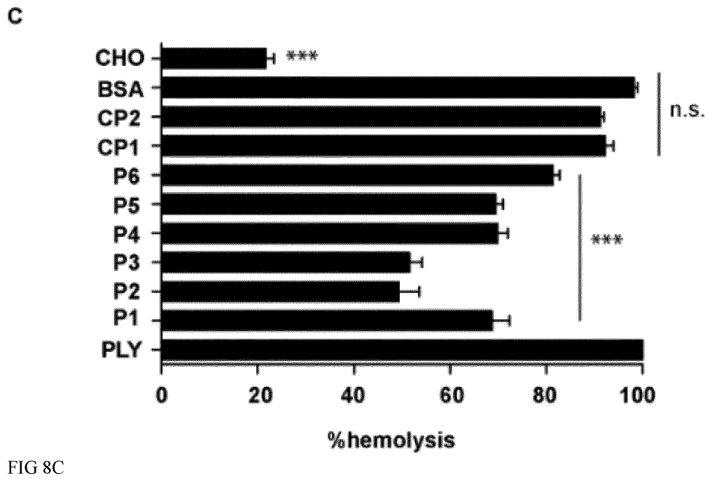

The inventors showed that MRC-1 serves as a common receptor for bacterial CDCs (Example 1). The inventors were able to determine the specific site of interaction between MRC-1 and the CDCs in the CTLD4 domain of MRC-1, one of the eight C-type lectin domains present in MRC-1, see FIG. 8A. The CTLD4 was shown to interact with the highly conserved CDC tryptophan rich-undecapeptide loop in domain 4 that also has the function of binding to membrane cholesterol, a defining characteristic of CDCs (Example 2).

Since the results indicated that that MRC-1 interacts with the cholesterol binding loop of the CDCs, the inventors hypothesized that peptides from the region of interaction could inhibit cholesterol binding toxin interactions with eukaryotic cells in general. Overlapping peptides from the CTLD4 were constructed, and peptides P2 and P3 containing amino acids that interact with the cholesterol loop of the CDCs were found to bind to CDCs and inhibit their induction of hemolysis, induction of inflammatory responses and cytolysis of macrophages (Example 3).

Using 2D primary cell culture and 3D lung tissue models, the inventors demonstrated that the peptides reduce toxin-induced epithelial damage, inflammation and bacterial invasion across the epithelium (Examples 4 and 5). Finally, the inventors used calcium phosphate nanoparticles (NPs) as peptide nanocarriers for intranasal delivery and show that co-administration of MRC1-peptide-conjugated NPs enhance survival and bacterial clearance in both mouse and zebrafish models of pneumococcal infection (Example 6).

### Medical Uses

Thus, in a first aspect, the present invention provides a polypeptide or peptidomimetic comprising:
a sequence of at least 7 residues differing by residue substitutions, deletions or insertions numbering 0-2 compared to the sequence:

(SEQ ID NO: 1)
GLTYGSPSEGFTWSDGSPVSYENWAYGEPNNYQNVEYCGELKGDPTMSW
NDINCEHLNNWICQ,

for use as a medicament.

The first aspect also encompasses a method of treatment, comprising administering the above polypeptide or peptidomimetic to a subject in need thereof. The first aspect further encompasses the use of the above polypeptide or peptidomimetic in the manufacture of a medicament.

The polypeptide or peptidomimetic of the first aspect may be for use in the treatment or prevention of a bacterial infection by a pathogen expressing a cholesterol-dependent cytolysin (CDC). The polypeptide or peptidomimetic may be for use in neutralizing bacterial CDCs in a subject.

The polypeptide or peptidomimetic may be for use as a medicament in the treatment or prevention of pneumococcal disease, preferably a pneumococcal disease selected from pneumococcal sinusitis, pneumococcal otitis, pneumococcal pneumonia and invasive pneumococcal disease including but not limited to pneumococcal sepsis and pneumococcal meningitis, preferably invasive pneumococcal disease.

The polypeptide or peptidomimetic may be for use in the treatment or prevention of secondary bacterial pneumonia following a virus infection.

The polypeptide or peptidomimetic may be for use in the treatment or prevention of streptococcal diseases such as tonsillitis, pneumonia and other respiratory tract infections, septicaemia, skin infections, necrotizing fasciitis, as well as against infections caused by Listeria bacteria, such as listeriosis or septicaemia, or by other disease caused by bacteria forming cholesterol-binding toxins.

The polypeptide or peptidomimetic may be for use in the prevention of carriage of streptococci, in particular Group A streptococci such as S. pyogenes. The polypeptide or peptidomimetic may be for use in the treatment or prevention of infections caused by Group A streptococci such as S. pyogenes.

The use may involve intranasal administration of the polypeptide or peptidomimetic to a subject in need thereof. Alternative routes of administration include peroral, inhalation, intramuscular, subcutaneous or intravenous administration of the polypeptide or peptidomimetic to the subject.

The use may further involve administration of antibiotics to the subject. The antibiotic may be administered before, concomitantly, or after administering the polypeptide or peptidomimetic of the present invention.

The polypeptide or peptidomimetic referred to in the first aspect may be formulated as a composition according to the second aspect described below.

### Nanoparticle Composition

In a second aspect, the present invention relates to a composition, comprising a polypeptide or peptidomimetic comprising:
a sequence of at least 7 residues differing by residue substitutions, deletions or insertions numbering 0-2 compared to the sequence:

(SEQ ID NO: 1)
GLTYGSPSEGFTWSDGSPVSYENWAYGEPNNYQNVEYCGELKGDPTMSW
NDINCEHLNNWICQ,

wherein the polypeptide or peptidomimetic is immobilized on a carrier, preferably a solid carrier, most preferably a particulate carrier.

The carrier may a nanoparticle (NP), preferably a calcium phosphate (CaP) nanoparticle.

Preferably, the carrier (such as CaP nanoparticles) contains 10-1000 mg peptide/g carrier, more preferably 50-500 mg peptide/g carrier, even more preferably 50-300 mg/g carrier, yet more preferably 50-200 mg/g carrier, most preferably about 75-150 mg/g carrier.

The primary particle size of preferred carrier nanoparticles for use with the invention ranges from 5 to 100 nm (most preferred 10-20 nm). The agglomerate size of such nanoparticles in water may range from 50 to 1000 nm (preferably 200-700 nm, most preferably 100-500 nm).

Nanoparticles have been proposed to be employed as carriers in vaccines due to their small size that facilitates their interaction with cells and other biological entities and they can both stabilize vaccine antigens and act as adjuvants (Al-Halifa et al. (2019) Nanoparticle-Based Vaccines Against Respiratory Viruses. Front. Immunol. 10:22). Both polymeric-based and inorganic-based nanoparticles are known in the vaccine field and are useful with the present invention. Among them, inorganic nanoparticles such as calcium phosphates represent a preferred material as nano-vaccine carrier/adjuvant. Other suitable inorganic materials for nanocarrier particles include gallium oxide, gallium phosphate, gold, iron oxide and silica. Among polymeric-based nanoparticles may utilize polymers including poly(α-hydroxy acids), poly(amino acids), or polysaccharides to create a vesicle which can accommodate the peptides. The most commonly used poly(α-hydroxy acids) for preparing polymeric NPs are either poly(lactic-co-glycolic acid) (PLGA) or poly(lactic acid) (PLA) which are often synthesized using a double emulsion-solvent evaporation technique. Alternatives include poly(amino acids) such as poly(γ-glutamic acid) (γ-PGA), poly(ε-lysine), poly(L-arginine), or poly(L-histidine) which do not require an emulsion step. These amphiphilic copolymers self-assemble via hydrophobic interactions to form polymeric structures consisting of a hydrophobic core and a hydrophilic outer shell.

For the present invention, preferred nanoparticles are larger agglomerates in the range of 100-500 nm that consist of several smaller primary particles (each primary particle in the range of 10-20 nm). Due to their synthesis route these particles have a fractal-like geometry, typical values of fractal dimension are 1.8-2.1. That means that they have a rather “open” structure that allows for the loading of the peptide throughout the whole agglomerate. This morphology is useful for the “protection” of the peptide from enzymatic degradation.

Calcium phosphate (CaP) nanoparticles (preferred) exhibit great potential as vaccine carrier/adjuvant (Yahua Lin et al (2017) Calcium phosphate nanoparticles as a new generation vaccine adjuvant, Expert Review of Vaccines, 16:9, 895-906). CaP is highly biocompatible because it occurs naturally in the human body (bones, tendons, teeth). Furthermore, calcium or inorganic phosphate ions exist in the bloodstream in the concentration range of 1-5 millimoles per liter. CaP per se belongs to the category of Generally Regarded as Safe as reported by the US Food and Drug Administration. Furthermore, CaP are biodegradable and biocompatible, and thus are safer than aluminum salt adjuvants. They elicit less production of IgE, milder local irritation, and inflammatory reaction than aluminum salt adjuvants.

There are several useful ways of conjugating a biomolecule (such as a peptide) on carriers that can be broadly distributed in two categories, (i) covalently, (ii) non-covalently. In the first category, the carrier surface is modified accordingly to allow for the chemical bonding through appropriate reactions with one functional group from the biomolecule, most often an amine ending or a carboxyl ending. This results in a rather firm conjugation of the biomolecule on the carrier surface. The second category involves the conjugation of the biomolecules by physical adsorption, or physisorption, that results in a rather stochastic self-assembly of the biomolecules on the carrier surface. Both covalent and non-covalent binding of the peptide of the invention to the carrier are contemplated. Physical adsorption is the preferred mode of associating the peptide with the carrier.

### Polypeptide or Peptidomimetic

In a third aspect, the present invention provides a polypeptide or peptidomimetic comprising:
a sequence of at least 7 residues differing by residue substitutions, deletions or insertions numbering 0-2 compared to the sequence:

(SEQ ID NO: 1)
GLTYGSPSEGFTWSDGSPVSYENWAYGEPNNYQNVEYCGELKGDPTMSW
NDINCEHLNNWICQ.

### Properties of the Polypeptide or Peptidomimetic

The polypeptide or peptidomimetic referred to in the first, second and third aspects above may comprise the following features.

### Sequence Features

The sequence of the polypeptide or peptidomimetic referred to in the first, second and third aspects may comprise YEN residues aligning with the bolded sequence at positions 21-23 of SEQ ID NO: 1. The sequence may comprise a T residue aligning with the position 12 of SEQ ID NO: 1. The sequence may comprise a S residue aligning with the position 14 of SEQ ID NO: 1.

The polypeptide or peptidomimetic may preferably comprise the sequence VSYENWA (SEQ ID NO: 4).

The polypeptide or peptidomimetic may comprise the sequence FTWSDGSPVSYEN (SEQ ID NO: 2), or a subsequence thereof being at least 5 residues long, preferably at least 6, more preferably at least 7, yet more preferably at least 8, still more preferably at least 9, even more preferably at least 10, further still preferably at least 11, most preferably at least 12 residues.

The polypeptide or peptidomimetic may comprise YENWAYGEPNNYQ (SEQ ID NO: 3), or a subsequence thereof being at least 5 residues long, preferably at least 6, more preferably at least 7, yet more preferably at least 8, still more preferably at least 9, even more preferably at least 10, further still preferably at least 11, most preferably at least 12 residues.

The polypeptide or peptidomimetic may comprise a consecutive sequence of at least 8, preferably at least 9, more preferably at least 10, yet more preferably at least 11, still more preferably at least 12, most preferably at least 13 residues differing by residue substitutions, deletions or insertions numbering 0-2 compared to the sequence SEQ ID NO:1.

Preferably, the polypeptide or peptidomimetic comprises a consecutive sequence of no more than 60 residues having more than 80% sequence identity to SEQ ID NO:1, preferably no more than 50, more preferably no more than 40, yet more preferably no more than 30, most preferably no more than 20.

The identification of the active site in the MRC-1 protein by the inventors allows designing a therapeutically effective polypeptide or peptidomimetic that is substantially shorter than the native MRC-1 protein. The shorter fragment is easier and cheaper to manufacture. It also has higher potency, at least on weight basis compared to the native MRC-1 protein sequence.

Thus, preferably, the polypeptide or peptidomimetic comprises no more than 100 residues in total, preferably no more than 60 residues, preferably no more than 50 residues, more preferably no more than 40 residues, yet more preferably no more than 30 residues, still more preferably no more than 20 residues, most preferably no more than 15 residues.

The residue substitutions, deletions or insertions may number 2 in total. Preferably, the residue substitutions, deletions or insertions may number 1 in total. Most preferably, the residue substitutions, deletions or insertions number 0 in total.

Preferably, the deletions or insertions number 0 in total.

The sequence may comprise at least one difference compared to any naturally occurring sequence and/or the polypeptide or peptidomimetic comprises a non-naturally occurring chemical moiety.

### Structural Features

Preferably, the polypeptide or peptidomimetic is a polypeptide. Alternatively, the polypeptide or peptidomimetic is a peptidomimetic.

The polypeptide or peptidomimetic may have a modified C-terminal, such as an amidated C-terminal. The polypeptide or peptidomimetic may have a modified N-terminal, such as an acylated N-terminal.

The polypeptide or peptidomimetic may have a cyclic backbone. The polypeptide or peptidomimetic may comprise one or more non-peptide bonds in the backbone.

The polypeptide or peptidomimetic may comprise one or more non-natural residues, preferably one or more D-amino acid residues. One or more the P residues may be hydroxylated.

The polypeptide or peptidomimetic may be conjugated to a detectable marker, preferably biotin, a fluorescent marker, or a radioactive label.

The polypeptide or peptidomimetic may be conjugated to a chemical moiety increasing residence time in plasma, such a polyethylene glycol, PAS-group, albumin, or the like.

### Functional Features

In order to be useful in the contemplated applications, the polypeptide or peptidomimetic referred to in the first, second and third aspects should preferably have certain functional properties.

The polypeptide or peptidomimetic may bind to PLY, SLO or LLO, preferably PLY. The polypeptide or peptidomimetic preferably binds to the cholesterol binding loop of bacterial cholesterol-dependent cytolysins, preferably Pneumolysin (PLY), streptolysin (SLO) or listeriolysin (LLO), most preferably PLY.

Preferably, the polypeptide or peptidomimetic is inhibitory to hemolysis by PLY, SLO or LLO, most preferably PLY. Preferably, the polypeptide or peptidomimetic is inhibitory to such hemolysis with an IC50 of less than 1000 μM, preferably 100 μM, more preferably 10 μM.

Preferably, the polypeptide or peptidomimetic is inhibitory to hemolysis by PLY with an IC50 of less than 1000 μM (preferably 100 μM, more preferably 10 μM) in an assay using blood diluted 1:100 in physiological phosphate-buffered saline combined with 1 μg/ml purified PLY at 37 C for 1 h.

Preferably, the polypeptide or peptidomimetic competitively interferes with the cholesterol-binding activity of PLY, SLO or LLO, preferably PLY. Preferably, the polypeptide or peptidomimetic is a competitive inhibitor with an IC50 of less than 1000 μM, preferably 100 μM, more preferably 10 μM.

### Discussion Concerning the Present Invention

The inventors developed peptides derived from the CTLD4 domain of the human mannose receptor, MRC-1, that interacts with the conserved cholesterol binding loop of CDCs, which is critical for toxin binding to eukaryotic cells. Out of the 8 peptides screened, two peptides showed the highest binding to purified CDCs and protected host cells against toxin-induced cytolysis and inflammation. It was shown that the peptides were effective against the three major bacterial CDCs, PLY, LLO and SLO. The peptides also bound in situ to PLY-producing pneumococci and blocked hemolysis, suggesting that peptides target both secreted and pneumococcal-localized PLY, since PLY has also been shown to be exposed on bacterial surfaces. Since CDCs are one of the major conserved bacterial virulence factors, targeted neutralization of their effects is an innovative approach to eliminate the bacteria without killing them, and hence the risk for resistance development is lower.

Besides inducing cytolysis, CDCs have been shown to promote bacterial invasion and entry into host cells in a dynamin-and F-actin-dependent manner. The inventors found that certain MRC-1 peptides effectively block bacterial invasion in the 3D lung epithelial model, as well as in primary human DCs. The ability of the peptides to reduce bacterial invasion is similar as blockage of PLY using antibodies. The human macrophage and DC receptor MRC-1 has been shown to promote bacterial uptake into phagosomes without culminating in lysosomal fusion, thereby providing a safe intracellular niche for bacteria. Since the peptides are derived from the CTLD4 domain of MRC-1 that interacts with the CDCs, the inventors hypothesize that they block bacterial uptake mediated by PLY-MRC-1 interaction. In DCs treated with the MRC-1 peptide, intracellular pneumococci did not co-localize with MRC-1, but co-stained with the autophagy marker LC3B, indicating that the bacteria are targeted to autophagy killing. Many bacterial pathogens avoid immune clearance by persisting intracellularly within host cells and contribute to the relapse of the infection. Hence, the peptides are useful to eliminate bacteria that have escaped antibiotic killing by persisting intracellularly within MRC-1-positive tissue macrophages and DCs.

Since peptides are generally prone to enzymatic degradation in vivo, the inventors developed CaP NPs loaded with an MRC-1 peptide that allowed rapid and efficient targeting to the lungs after intranasal administration. CaP NPs are non-toxic and have been successfully used to deliver bioactive molecules like peptides and microRNA owing to their cellular permeability.

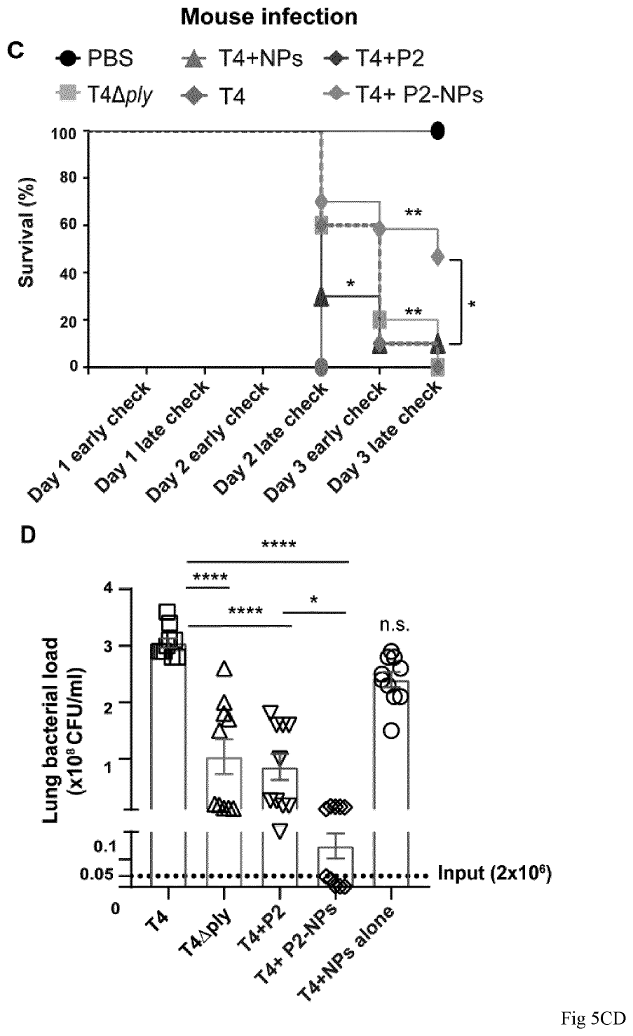

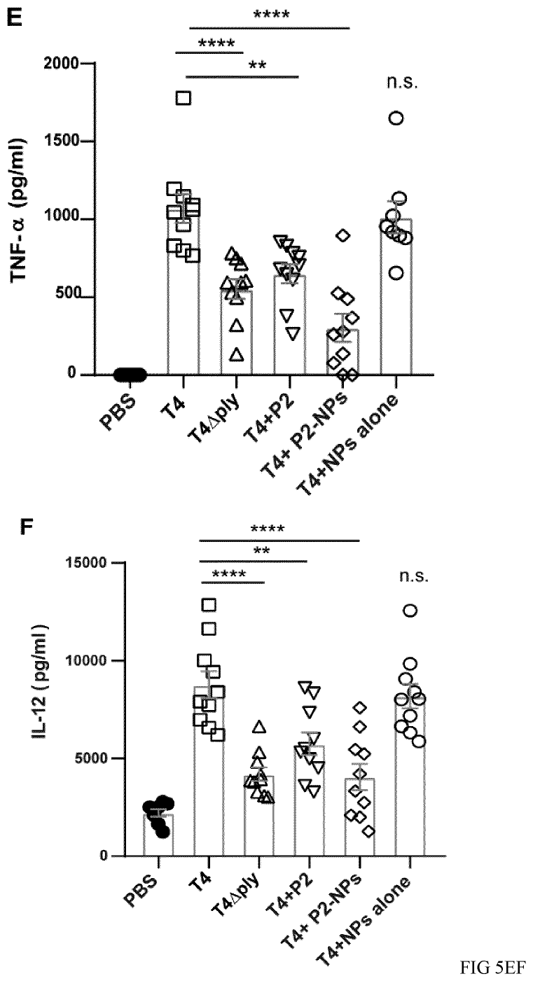

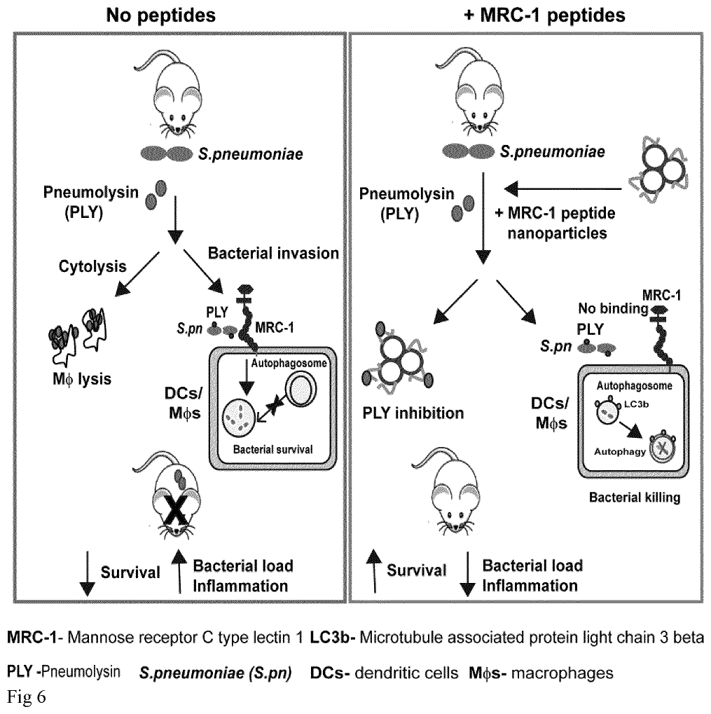

In conclusion, the experimental data reveal that certain MRC-1 derived peptides bind to bacterial pore-forming toxins, inhibit lysis of host macrophages, and reduce inflammation. They also block MRC-1 mediated bacterial uptake into DCs and promote autophagy killing of intracellular bacteria. Using two in vivo models, zebrafish and mice, the inventors demonstrated that administration of peptide-conjugated NPs enhanced survival against pneumococcal infection as well as reduced the bacterial load and inflammation in the lungs (FIG. 6). Thus, the inventors envisage that these toxin-binding peptides are useful (possibly in combination with antibiotics) to treat patients with bacterial infections to neutralize the cytotoxicity and inflammation induced by pore-forming toxins. Since severely ill influenza A virus infected patients often get secondary pneumonia caused by bacterial respiratory pathogens, primarily S. pneumoniae and S. pyogenes, shown to be present in the lung tissue of ca 40% of the lethal cases during the Spanish flu, intranasal delivery of peptide-coated NPs might be particularly useful in cases with acute respiratory distress syndrome where secondary pneumonia is suspected.

### General Aspects Relating to the Present Disclosure

The term “comprising” is to be interpreted as including, but not being limited to. All references are hereby incorporated by reference. The arrangement of the present disclosure into sections with headings and subheadings is merely to improve legibility and is not to be interpreted limiting in any way, in particular, the division does not in any way preclude or limit combining features under different headings and subheadings with each other.

### EXAMPLES

The following examples are not to be regarded as limiting. For further information on the experimental details, the skilled reader is directed to a separate section titled Materials and Methods.

### Example 1: MRC-1 Co-Localizes with the Bacterial CDCs, PLY, LLO and SLO, in Human Dendritic Cells

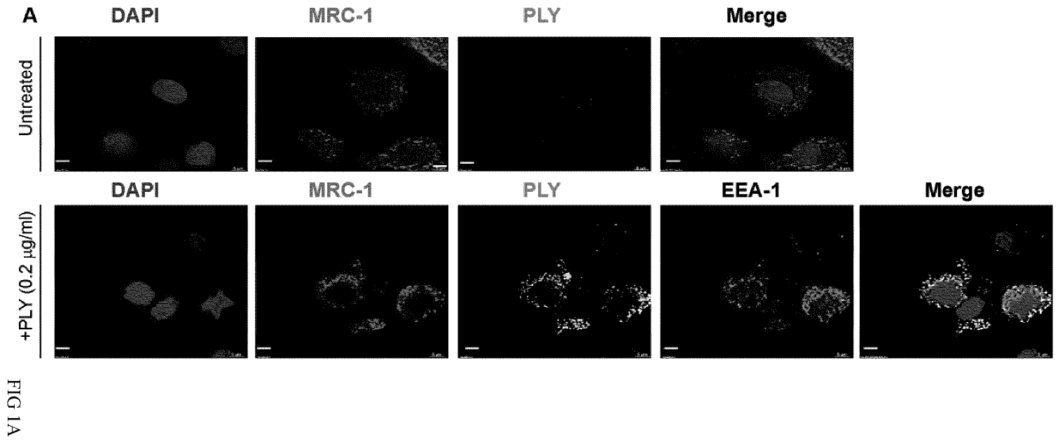

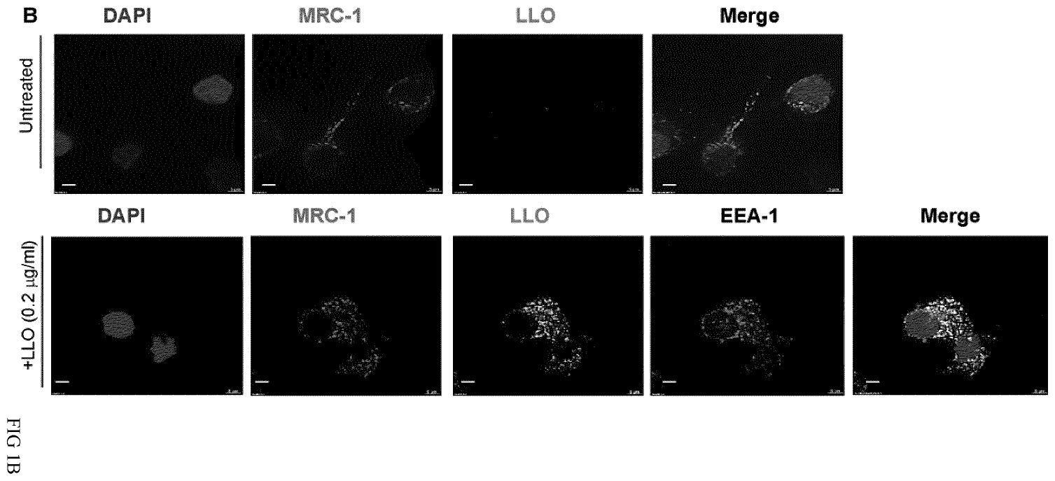

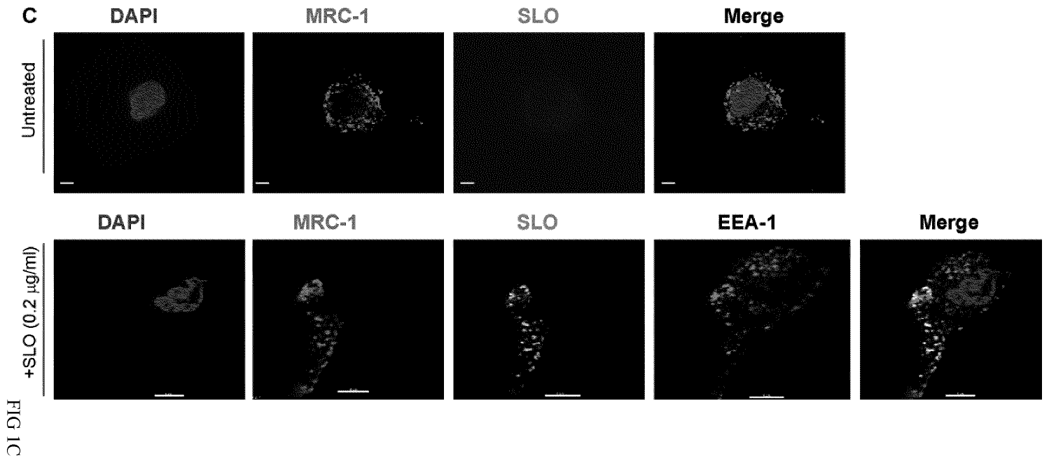

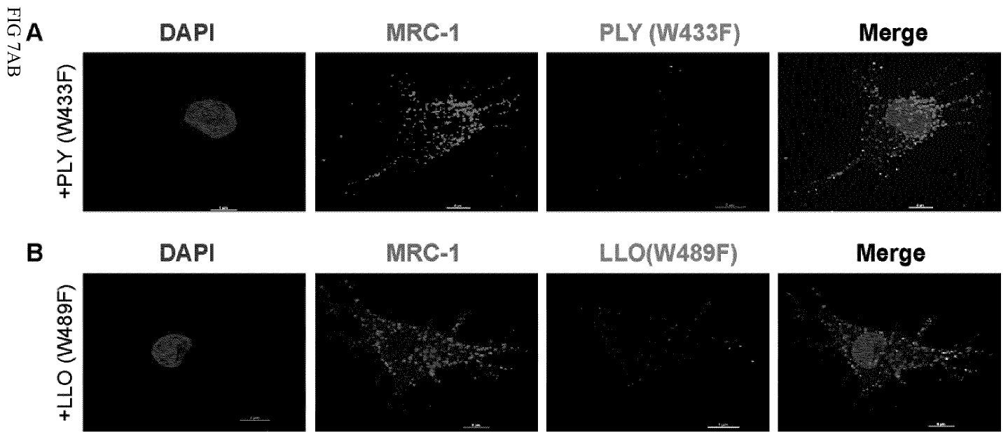

To test whether MRC-1 could serve as a common receptor for structurally conserved bacterial CDCs, the inventors incubated human monocyte-derived DCs with a non-cytolytic dose (0.2 μg/ml) of the purified toxins, PLY, LLO, and SLO, for 45 min, and performed immunofluorescence staining. The inventors found that MRC-1 co-localized with all the three CDCs in DCs (FIGS. 1A-C). To verify uptake by DCs, the inventors co-stained for the early endosomal antigen, EEA-1, and found that MRC-1 co-localized with the three CDCs along with EEA-1 (FIGS. 1A-C). To test whether the cholesterol binding loop in domain 4 of the CDCs, that binds to cholesterol on host cells, is also involved in the interaction with MRC-1, the inventors used toxoid derivates of PLY (W433F) and LLO (W489F) bearing point mutations in a key tryptophan residue of the cholesterol binding loop. In contrast to the wild-type toxins, the toxoids showed drastically reduced binding to the DCs and did not co-localize with MRC-1, indicating that the cholesterol binding loop of PLY and LLO is essential for the interaction with MRC-1 on DCs (FIGS. 7A, B).

### Example 2: The CTLD4 Domain of MRC-1 Interacts with the Cholesterol Binding Loop of Bacterial CDCs

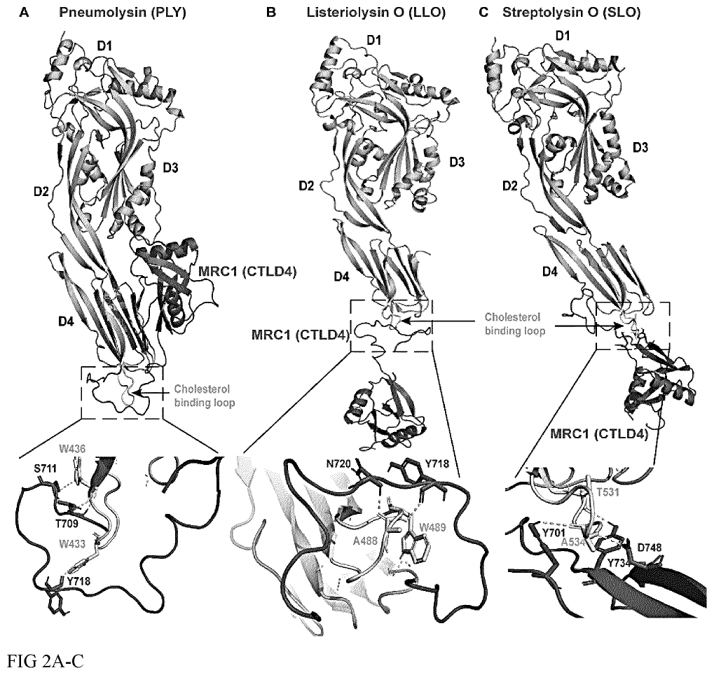

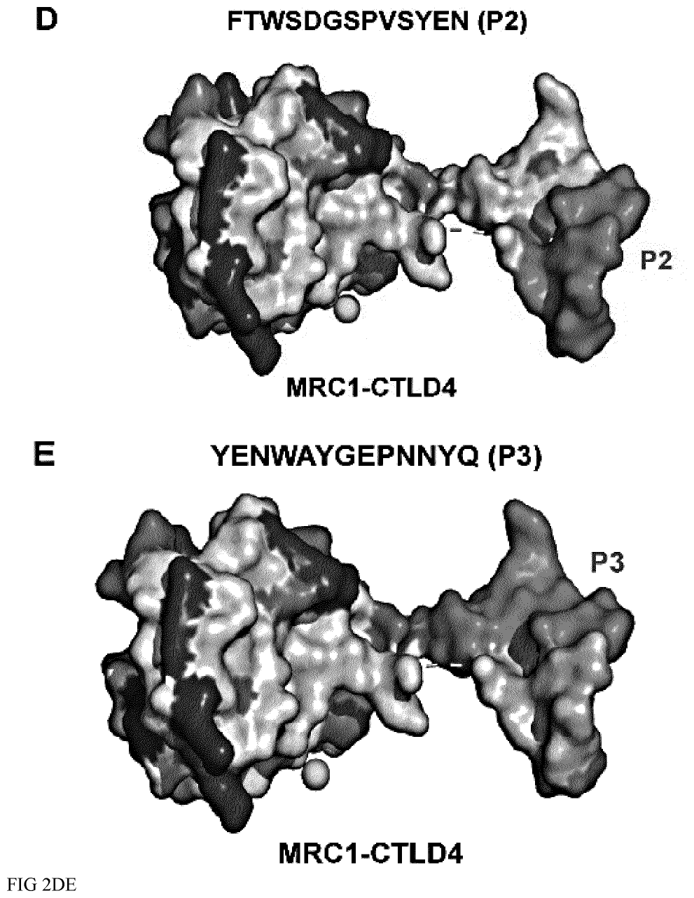

Next, the inventors wanted to determine the specific site of interaction between MRC-1 and the CDCs. The inventors recently showed that the CTLD4 domain of MRC-1 interacts with the membrane binding domain 4 of PLY. Hence, the inventors performed computational docking of the crystal structures of PLY, LLO and SLO with the CTLD4 domain of MRC-1 on the ClusPro 2.0 docking server, based on least energy configuration. The structures of PLY, LLO and SLO are conserved and consist of four domains, D1-D4, wherein domain 4 binds to cholesterol on the eukaryotic cell membrane (FIGS. 2A-C). The tryptophan rich-undecapeptide loop (highlighted in yellow) in domain 4 binds to the membrane cholesterol and is highly conserved amongst the CDCs. Results showed that the CTLD4 of MRC-1 (red) interacts with the cholesterol binding loop (yellow) in domain 4 of PLY, LLO and SLO, respectively (FIGS. 2A-C). Particularly, the inventors found that the tryptophan residues, W433 of PLY and W489 of LLO, located in the cholesterol binding loop of domain 4, are involved in polar interactions with CTLD4 of MRC-1. This was in line with the data showing that the mutant derivatives, PLY (W433F) and LLO (W489F), did not interact with MRC-1 (FIGS. 7A, B).

### Example 3: MRC-1 Peptides Bind to CDCs and Inhibit Their Induction of Hemolysis and Cytolysis of Macrophages

Since our results indicated that that MRC-1 interacts with the cholesterol binding loop of the CDCs, the inventors hypothesized that peptides from the region of interaction could inhibit toxin interactions with eukaryotic cells. Therefore, the inventors constructed overlapping 13-mer peptides from the CTLD4 of MRC-1, and two negative control peptides from the fibronectin type II domain and intracellular tail (FIG. 8A and Table 51). The peptides were commercially synthesized to >95% purity and screened for their ability to inhibit hemolysis of red blood cells induced by purified bacterial toxins. Addition of peptides P1-P6 inhibited PLY- and LLO-induced hemolysis as opposed to the control peptides, CP1 and CP2, as evident by the residual red blood cell pellet at the end of the hemolytic assay (FIG. 8B). Peptides P2 and P3 were the most potent, inhibiting hemolysis by up to 50% (FIG. 8C). In agreement, both peptides P2 and P3 contain amino acids that interact with the cholesterol loop of the CDCs (Table 51). The inventors found that both peptides, P2 and P3, were surface localized on the MRC-1 CTLD4 domain, which is ideal for interactions with bacterial toxins (FIGS. 2D, E). Cholesterol, a known inhibitor of PLY-induced hemolysis, was used as a positive control. Bovine serum albumin (BSA) was used as a negative control to verify that the inhibition of PLY-mediated hemolysis by the MRC-1 peptides was specific.

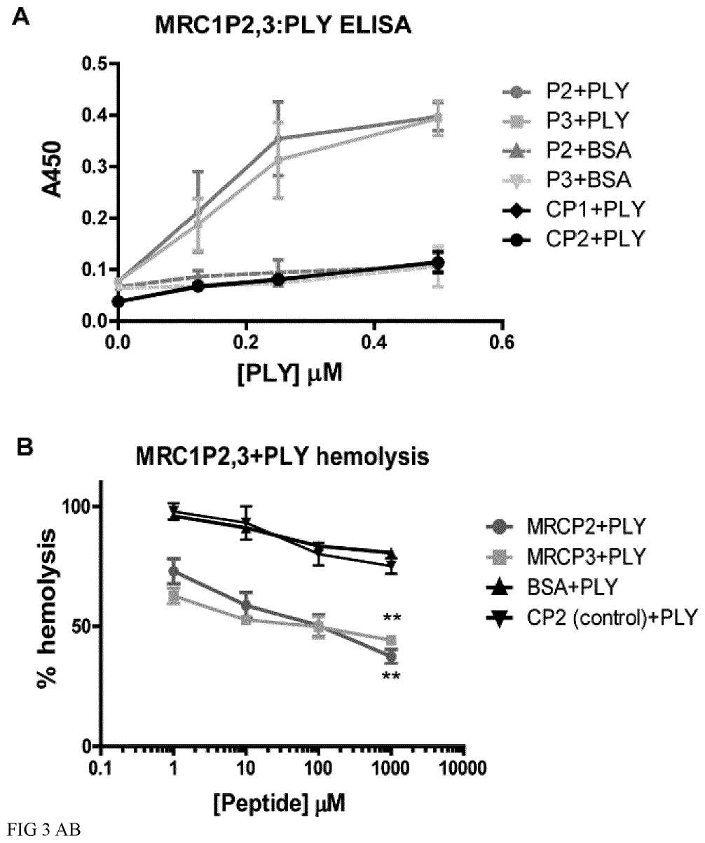

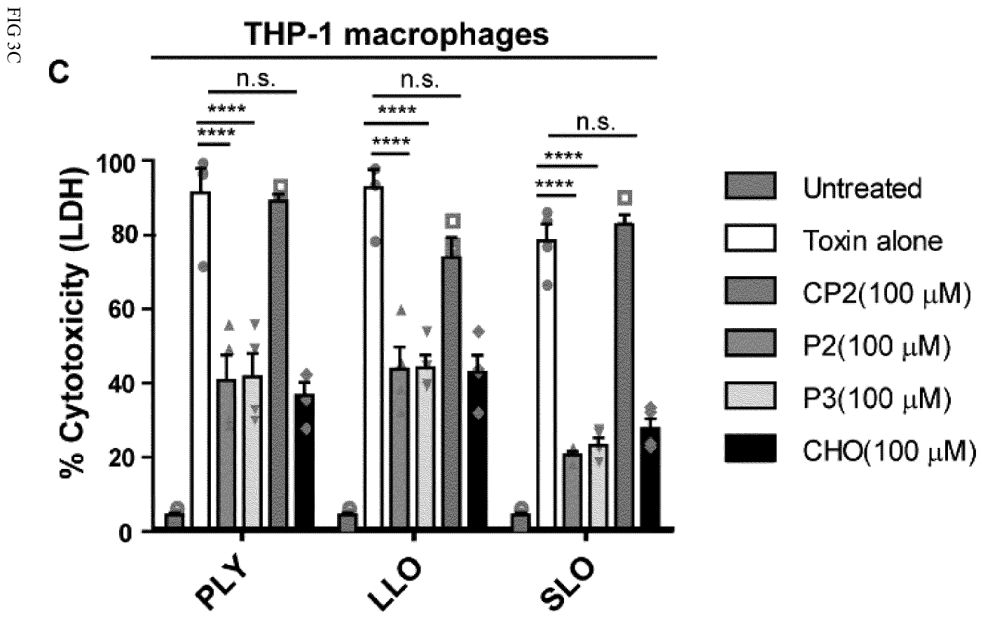

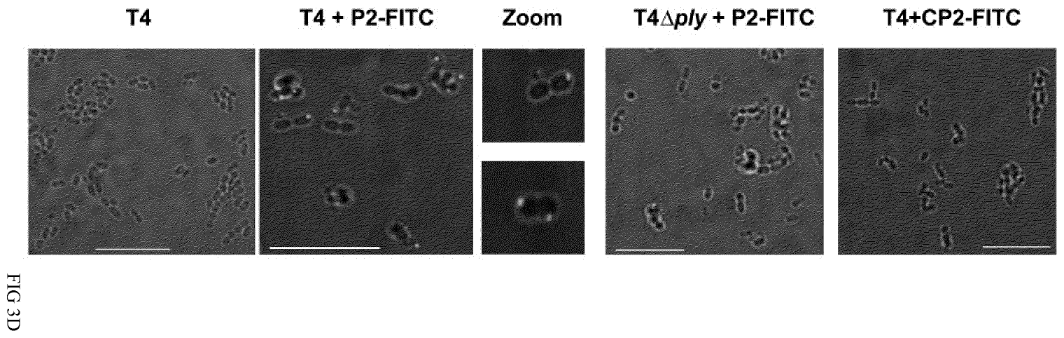

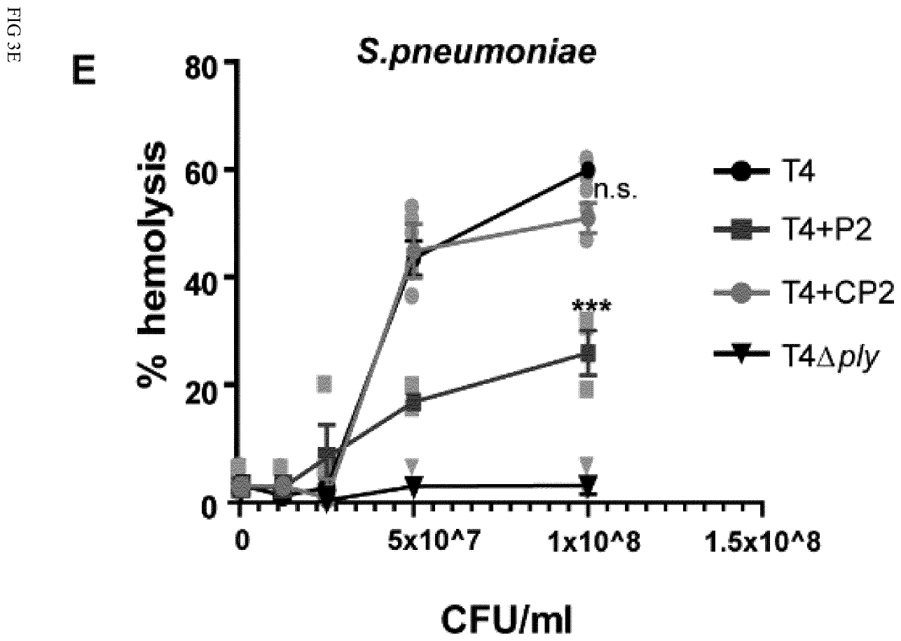

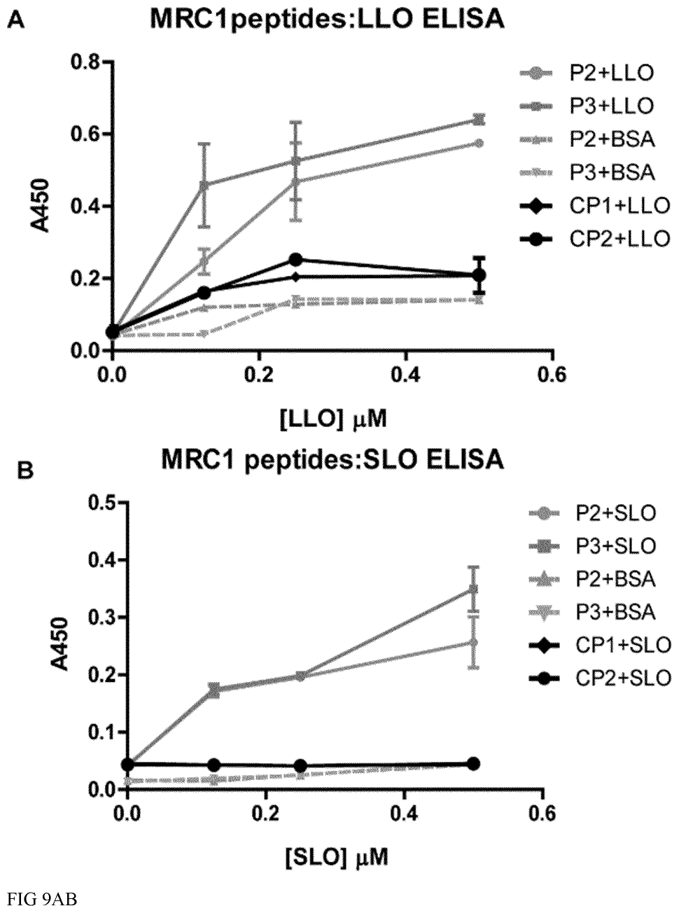

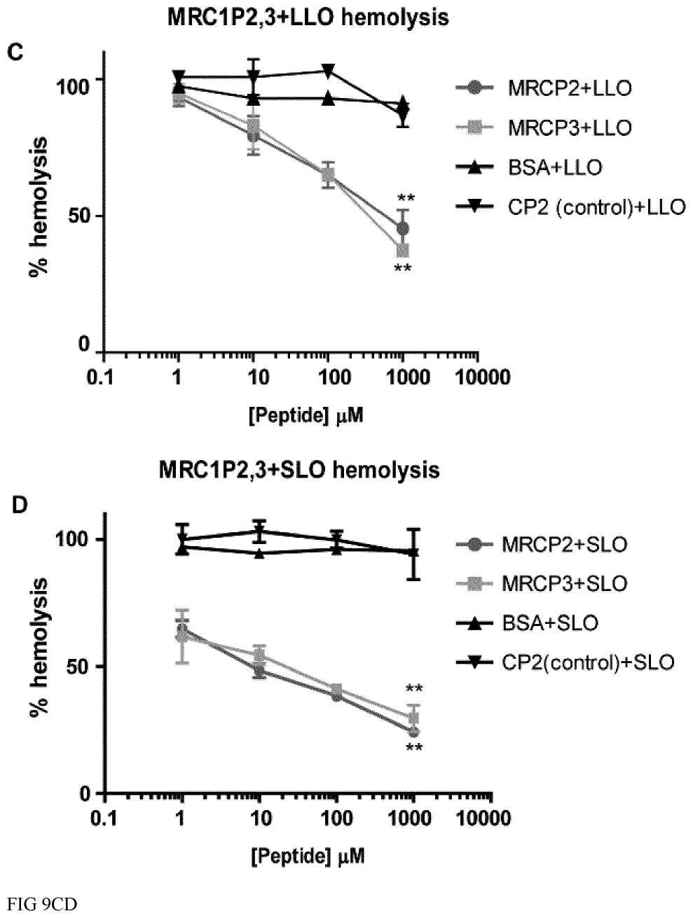

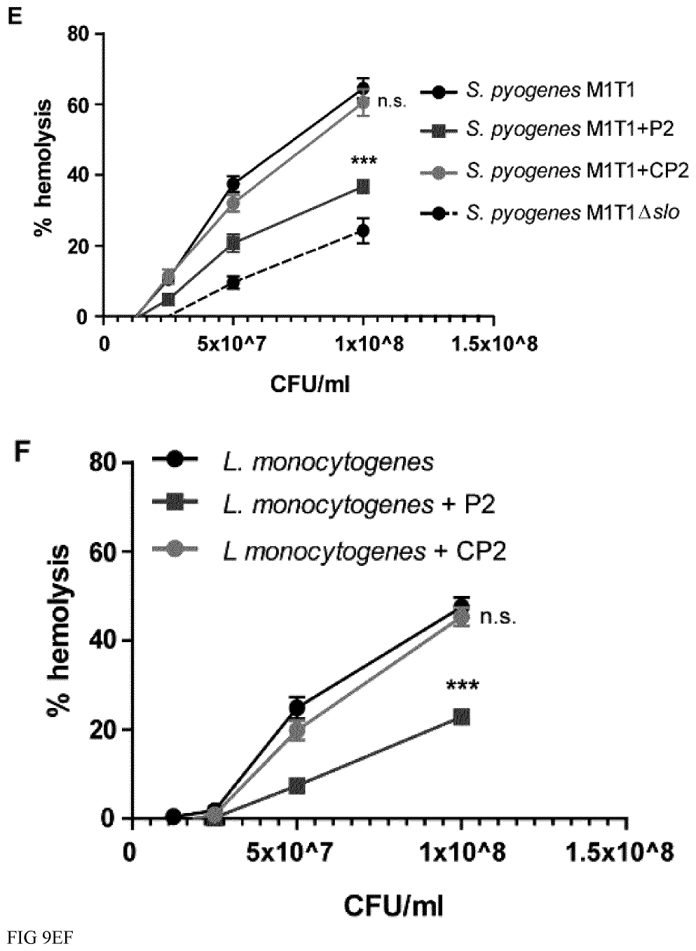

Then the inventors set up an ELISA to ascertain the binding of MRC-1 peptides to the purified CDCs and focused on the partially overlapping peptides P2 and P3 that showed the highest potency. Increasing doses of purified PLY or BSA (negative control) were added to peptides P2, P3, or control peptides CP1 or CP2, that were immobilized on the plates. Peptides P2 and P3 were found to bind dose-dependently to PLY, but not to BSA, suggesting that the binding was specific (FIG. 3A). The control peptides, CP1 and CP2, showed only background levels of binding. Peptides P2 and P3 also bound dose-dependently to LLO and SLO (FIGS. 9A, B). To determine the optimal working concentration of the peptides, the inventors performed a hemolysis assay by titrating increasing concentrations of the peptides in the presence of 1 μg/ml PLY. The inventors found that both peptides P2 and P3 dose-dependently inhibited PLY-induced hemolysis, resulting in up to 50% inhibition at 100 μM dose (FIG. 3B). Moreover, addition of 100 μM of P2 and P3 also inhibited LLO- and SLO-induced hemolysis by ˜35% and 60% respectively (FIGS. 9C, D).

To visualize inhibition of the bacterial CDCs by the peptides in real time, the inventors performed live imaging using human THP-1 monocyte-derived macrophages upon addition of PLY in the presence or absence of peptide P2. The cells were pre-loaded with the live-dead stain comprising of Calcein AM and propidium iodide that differentially stain live and dead cells green and red respectively. The cells were imaged for 20 min post addition of 0.5 μg/ml PLY in the presence or absence of 100 μM peptide P2, or control peptide CP2. In contrast to untreated cells, addition of PLY resulted in membrane blebbing and positive staining of cells by propidium iodide. However, in the presence of the peptide P2, but not of the control peptide CP2, most of the cells were intact and stained green, indicating protection from cytolysis. Cholesterol was used as positive control, and BSA as a negative control to show the specificity of the peptides. No cell death was observed when the toxoid form of PLY, Pdb (W433F), that is defective in pore-formation, was added. Peptide P2 also protected the cells from cytolysis mediated by LLO and SLO. To quantify cell death, the inventors measured the release of lactate dehydrogenase (LDH) from lysed cells into the culture supernatant. Peptides P2 and P3 significantly reduced cell death of macrophages induced by PLY, LLO or SLO (FIG. 3C), but no significant effect was observed with the control peptide CP2.

Next, the inventors tested whether the peptides could bind in situ to toxin-producing bacteria. The inventors incubated FITC-labelled peptides with the PLY-producing pneumococcal strain T4 (TIGR4 of serotype 4) and its isogenic PLY mutant, T4Δply, and analyzed by fluorescence microscopy. The inventors found that peptide P2 bound to T4, but not to the PLY deficient strain (FIG. 3D), suggesting that the peptides bind to PLY directly on the surface of the bacteria. However, the control peptide CP2 did not show any binding, indicating that the binding was specific. Moreover, the inventors measured the hemolytic activity of toxin-producing bacteria in the presence of 100 μM peptide P2 or CP2. The inventors found that in the presence of peptide P2, the hemolytic activity of the pneumococcal strain T4 was significantly reduced, while the control peptide CP2 showed no effect on the hemolytic activity (FIG. 3E). Peptide P2 also significantly reduced the hemolytic activity of the strains S. pyogenes type M1T1 and L. monocytogenes type 1 that express the toxins SLO and LLO respectively (FIGS. 9E, F).

TABLE S1
Sequence and domain location of MRC-1 peptides. The residues predicted to
interact with the cholesterol binding loop are highlighted in bold.

| Peptide | | Location on | |
|---|---|---|---|
| nomenclature | Sequence | MRC-1 | SEQ ID NO |
| P1 | 716-GLTYGSPSEGFTW-728 | CTLD4 | 5 |
| P2 | 726-FTWSDGSPVSYEN-738 | CTLD4 | 2 |
| P3 | 736-YENWAYGEPNNYQ-748 | CTLD4 | 3 |
| P4 | 746-NYQNVEYCGELKG-758 | CTLD4 | 6 |
| P5 | 756-LKGDPTMSWNDIN-768 | CTLD4 | 7 |
| P6 | 766-DINCEHLNNWICQ-778 | CTLD4 | 8 |
| CP1 | 181-DCTSAGRSDGWLW-193 | FN II domain | 9 |
| CP2 | 1444-LVGNIEQNEHSVI-1456 | Intracellular | 10 |
| | | tail | |

### Example 4: The MRC-1 Peptides Reduce Pro-Inflammatory Responses in Macrophages as Well as Cytotoxicity in a 3D Lung Tissue Model

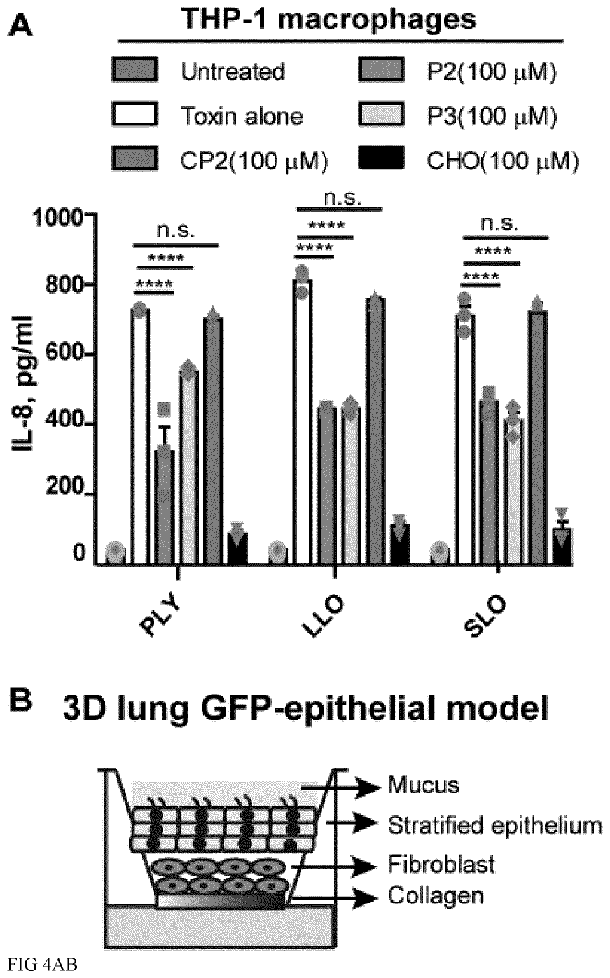

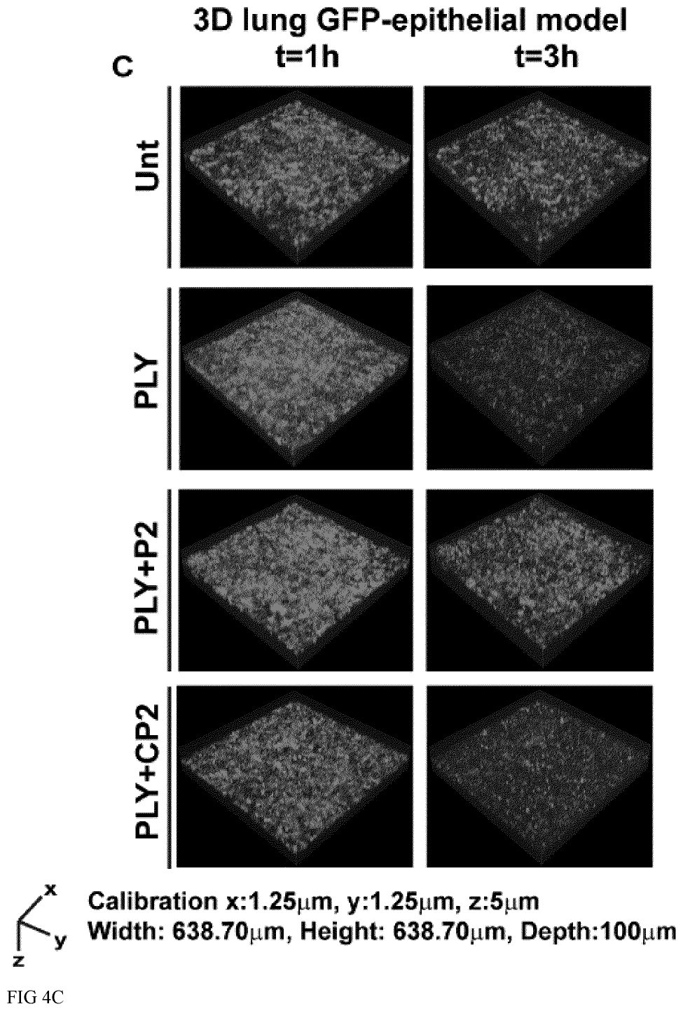

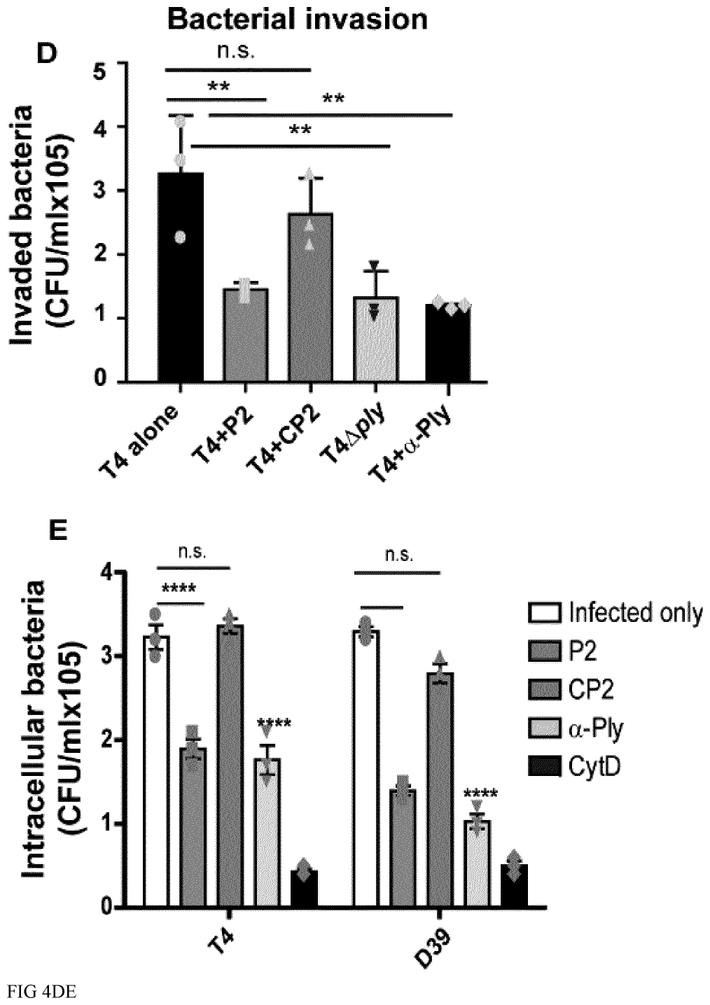

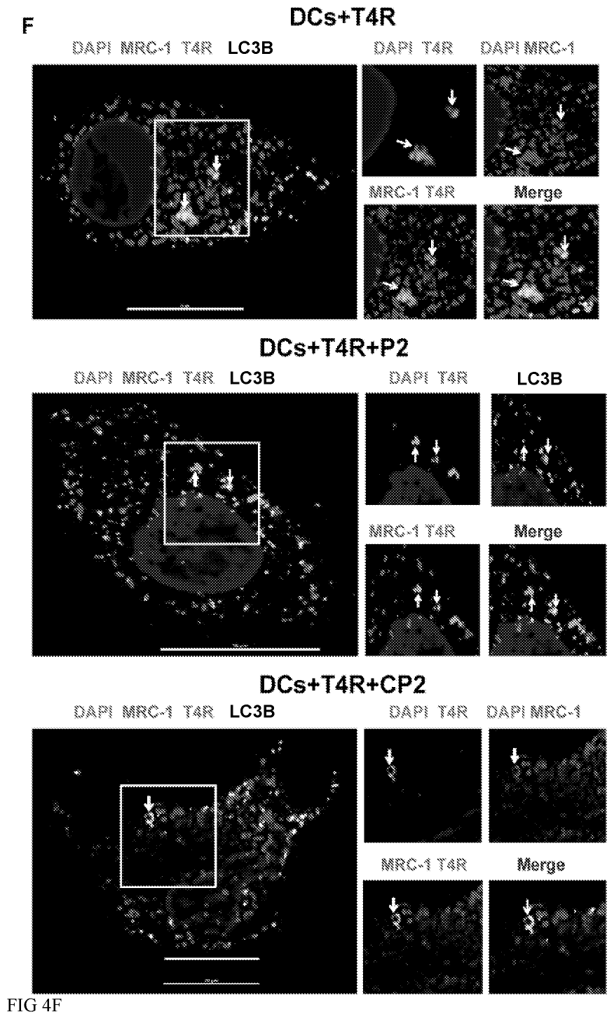

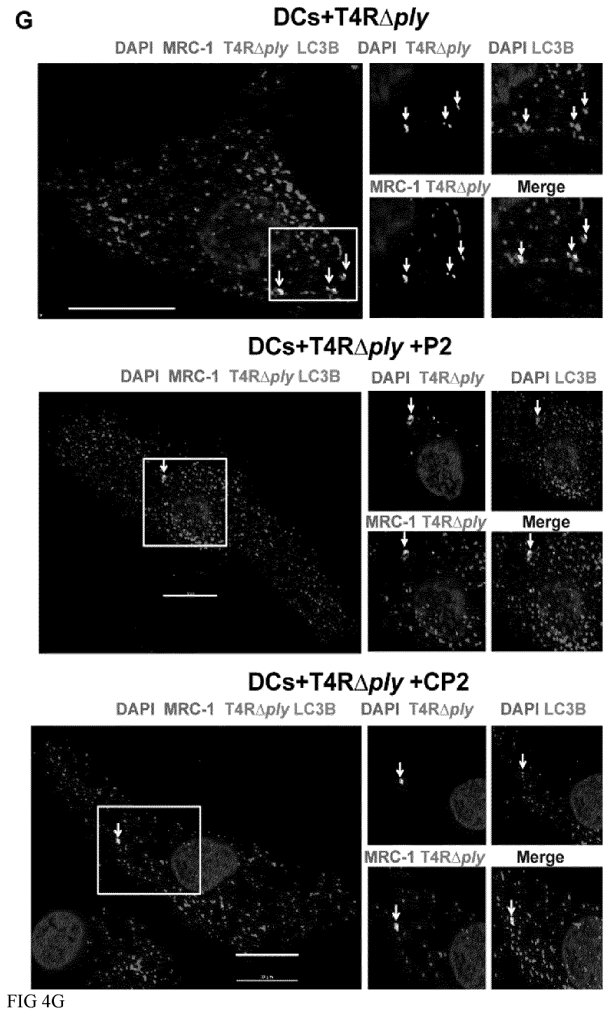

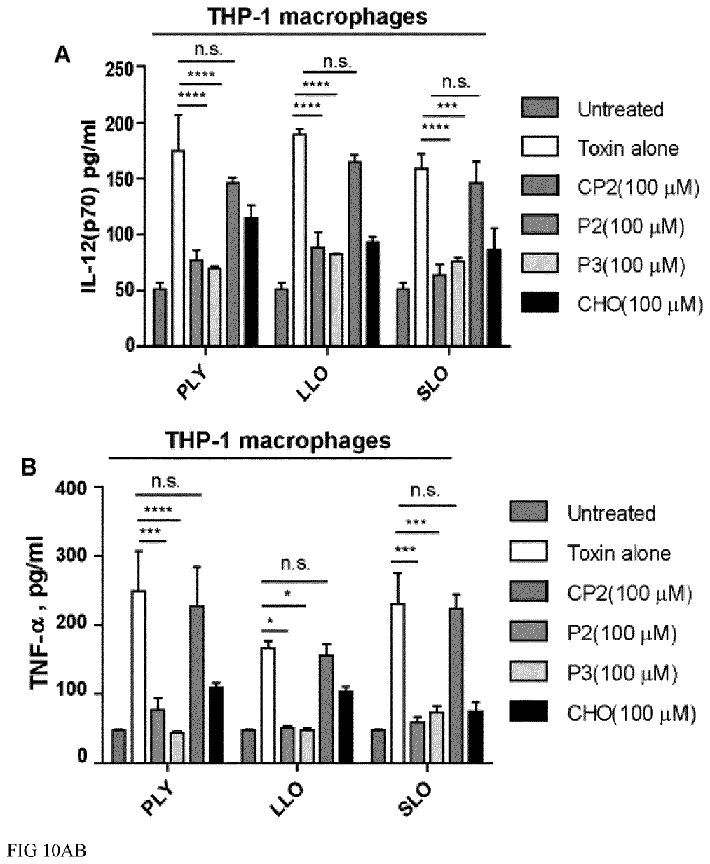

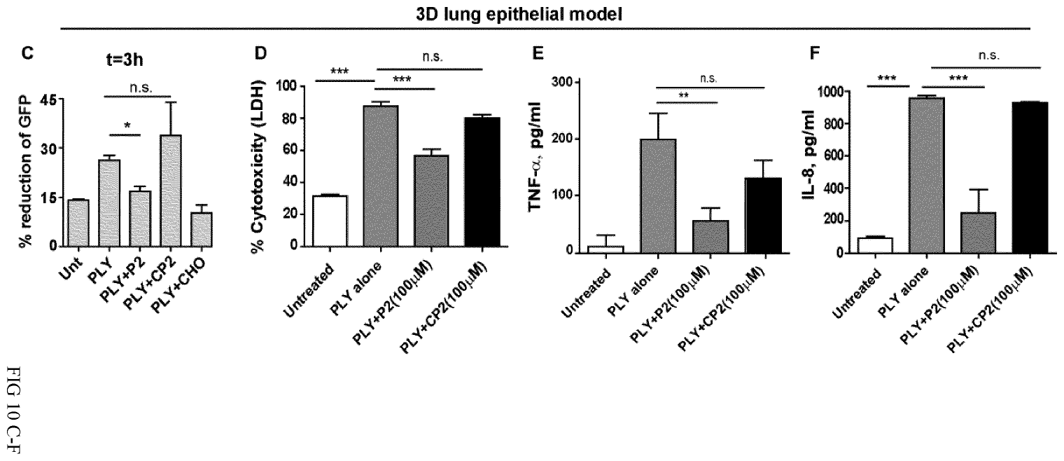

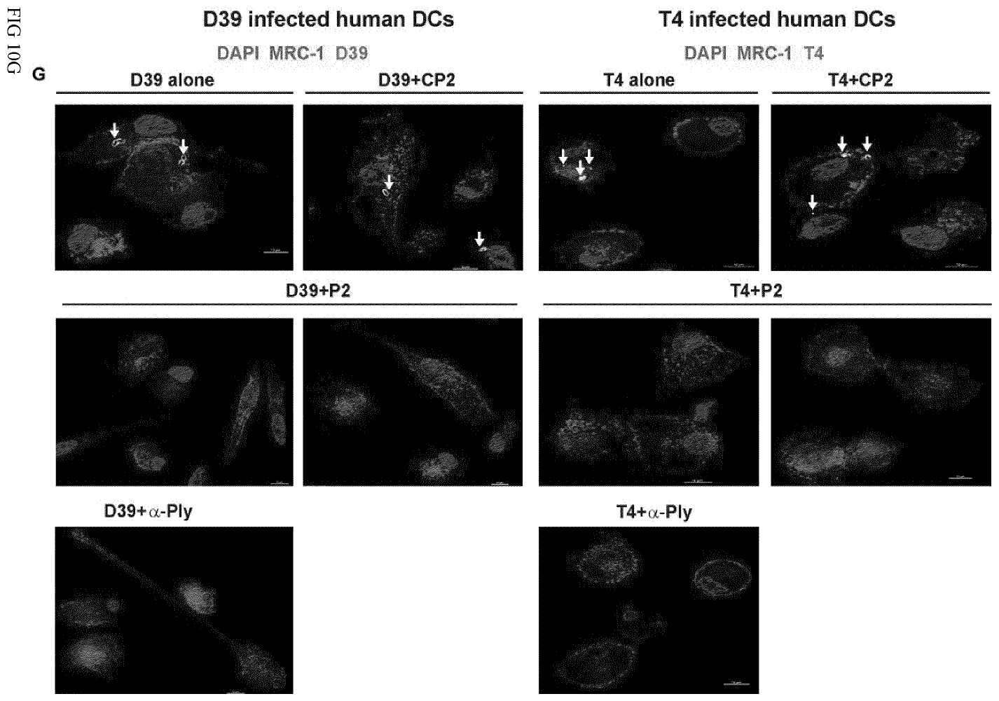

Bacterial CDCs, such as PLY, are known to induce a robust inflammatory response in human macrophages. Hence, the inventors next measured the release of pro-inflammatory cytokines by human THP-1 derived macrophages at 18 h post challenge with PLY, LLO or SLO (0.5 μg/ml) in the presence of 100 μM peptides P2 or P3, or control peptide CP2. The inventors found that in contrast to CP2, peptides P2 and P3 significantly reduced the release of the chemokine IL-8 and the pro-inflammatory cytokines TNF-α, and IL-12 (FIGS. 4A and 10A, B). Cholesterol was used as positive control.

To study toxin-mediated tissue pathology associated with pneumococcal pneumonia, the inventors utilized a green-fluorescent protein (GFP)-expressing 3D lung epithelial tissue model with an air-exposed stratified epithelial layer on top of lung fibroblast matrix layer (FIG. 4B). The fibroblasts and epithelial cells in the model were derived from human lung tissue and have been shown to mimic the lung physiological conditions like epithelial stratification, cilia and mucus secretion. The inventors performed live imaging to monitor the loss of GFP expression by the epithelial cells upon stimulation with 50 μg PLY in the presence or absence of 100 μM peptide P2 or control peptide CP2. The inventors found that at 3 h post challenge, PLY stimulation led to a greater reduction of the GFP signal compared to the untreated control, indicating epithelial disruption (FIG. 4C). The results were quantified in FIG. 10C. The loss of GFP expression was significantly lower in the presence of peptide P2 in comparison with the control peptide CP2. Cholesterol was used as positive control to inhibit epithelial damage by PLY. To quantify epithelial damage, the inventors measured LDH release into the supernatant at 18 h post challenge. Concurrent with the loss of GFP expression, PLY stimulation of the lung tissue model induced a robust release of LDH into the supernatant, which was significantly reduced in the presence of the peptide P2 (FIG. 10D). The control peptide CP2 did not show any significant reduction in LDH release compared to the PLY-treated model.

Epithelial cells in the respiratory tract constitute the primary barrier against pathogens and mediate innate immune response by producing antibacterial factors as well as secrete pro-inflammatory cytokines and chemokines to attract phagocytic cells to the site of infection. Excessive inflammatory responses cause tissue damage and thereby increase mortality of respiratory infections. Hence, the inventors measured the pro-inflammatory cytokine release in the lung tissue model in response to PLY with or without the peptides. The inventors found a significantly reduced release of the neutrophil chemokine IL-8 and of TNF-α upon addition of peptide P2, but not with peptide CP2 (FIG. 10E, F).

### Example 5: Toxin-Mediated Bacterial Invasion of the Lung Epithelium and Intracellular Bacterial Survival are Reduced by Treatment with MRC-1 Peptides

During invasive diseases such as pneumonia, bacteria breach the tight junctions of the epithelial barrier in order to invade underlying tissues and spread to sterile sites via the bloodstream. The inventors therefore investigated the role of CDCs in promoting bacterial invasion into the epithelium by counting the colony forming units (CFUs) upon infection with strainT4 or its isogenic PLY mutant (T4Δply) in our 3D lung epithelial model. The inventors found that the PLY mutant showed reduced invasion into the epithelium as compared to the wildtype T4 (FIG. 4D), suggesting that PLY promotes pneumococcal invasion of the epithelium. Addition of peptide P2, but not of CP2, significantly reduced the number of invading bacteria, implying that the peptide inhibits bacterial translocation across the epithelium by blocking PLY (FIG. 4D). The anti-PLY antibody was used as a positive control to demonstrate the role of PLY in pneumococcal invasion.

Besides cytolysis, PLY also promotes intracellular survival of pneumococci in DCs and lung alveolar macrophages, as well as trafficking across the blood brain barrier to invade the brain. Hence, the inventors measured the intracellular bacterial load of pneumococcal strains T4 of serotype 4 and D39 of serotype 2 in DCs, in the presence or absence of the PLY-binding peptide P2 at 3 h post infection. Addition of peptide P2 significantly reduced the number of intracellular bacteria in DCs (FIG. 4E), while the control peptide CP2 did not show any significant difference. The anti-PLY antibody was used as a control to verify the effect of PLY on intracellular pneumococcal survival. The actin polymerization inhibitor, cytochalasin D (CytD), was used as positive control to inhibit DC phagocytosis. The inventors also performed fluorescence microscopy to visualize intracellular bacteria in DCs in the presence of peptides P2 and CP2. The results agreed with the data from the CFU plating assay. Intracellular pneumococci (green) were detected in infected DCs that co-localized with MRC-1 (red) (FIG. 10G). DCs that were treated with peptide P2 or anti-PLY were devoid of intracellular bacteria.

A key strategy of phagocytic immune cells to eliminate intracellular bacteria is through autophagy. During autophagy, the bacteria are enclosed by double-membrane structure called phagophore. The autophagy protein microtubule-associated 1 light chain 3 (LC3) undergoes cleavage and associate with the phagophore upon lipidation. The autophagosomes fuse with lysosomes leading to degradation of intracellular bacteria. The mannose receptor, MRC-1, has been implicated in inhibiting phagosome maturation as well as fusion with lysosomes. The inventors therefore tested the effect of the PLY-inhibiting peptides on the fate of intracellular pneumococci by immunofluorescence microscopy. DCs were infected with the unencapsulated strain T4R (to get better uptake than with encapsulated bacteria) or its isogenic PLY mutant T4RΔply, and were immunostained for MRC-1, pneumococci (anti-serum), and the autophagy marker LC3B, at 3 h post infection.

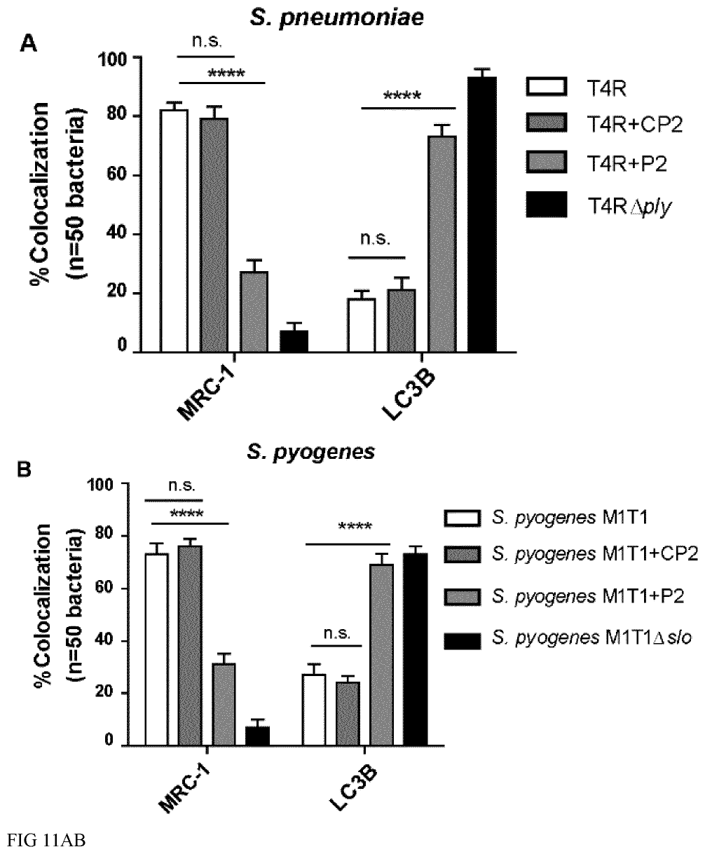

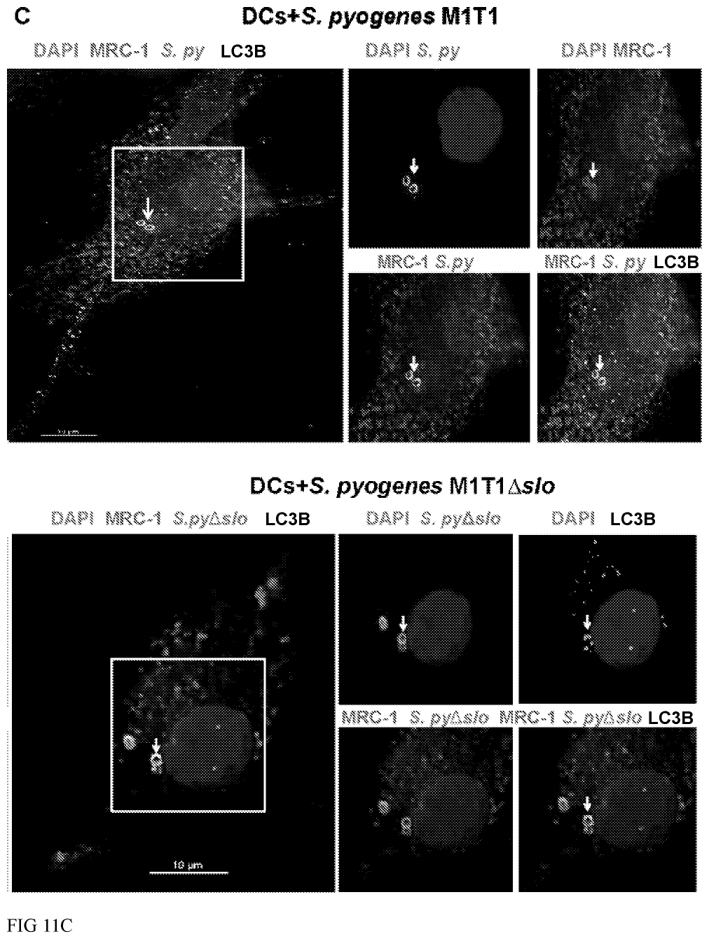

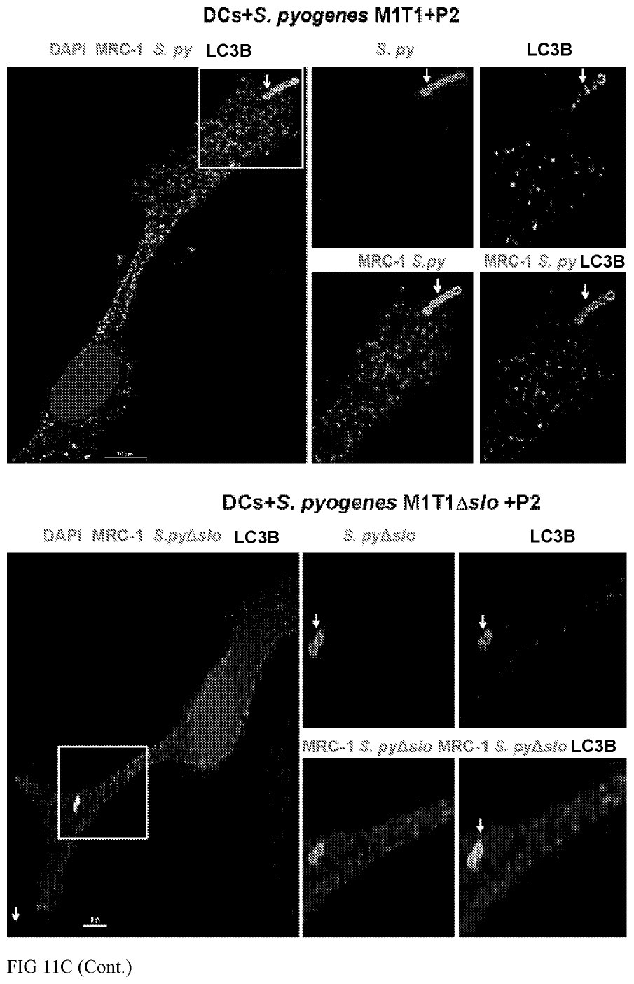

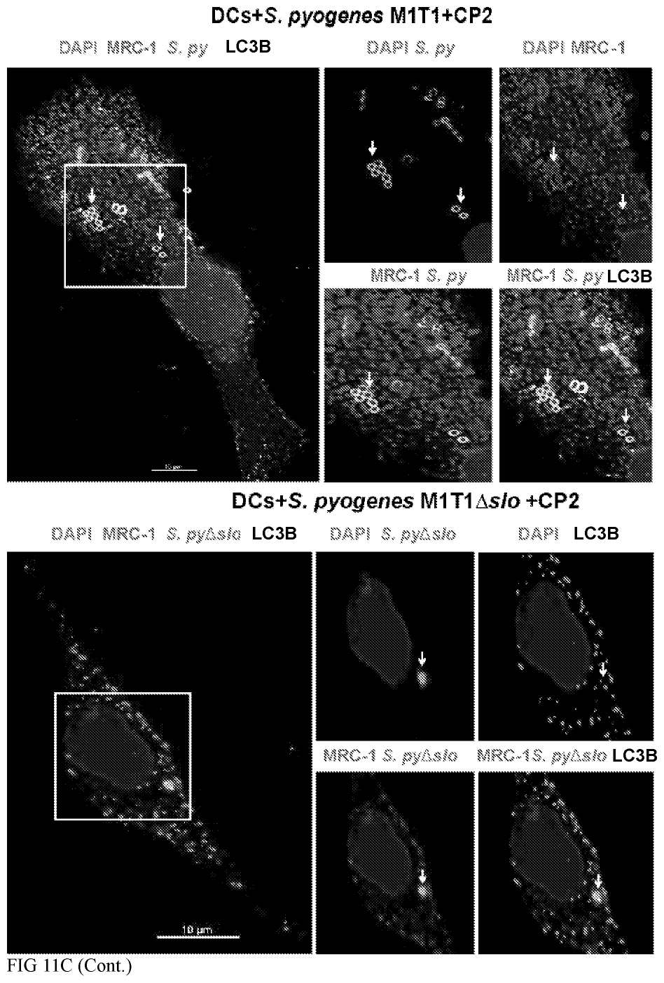

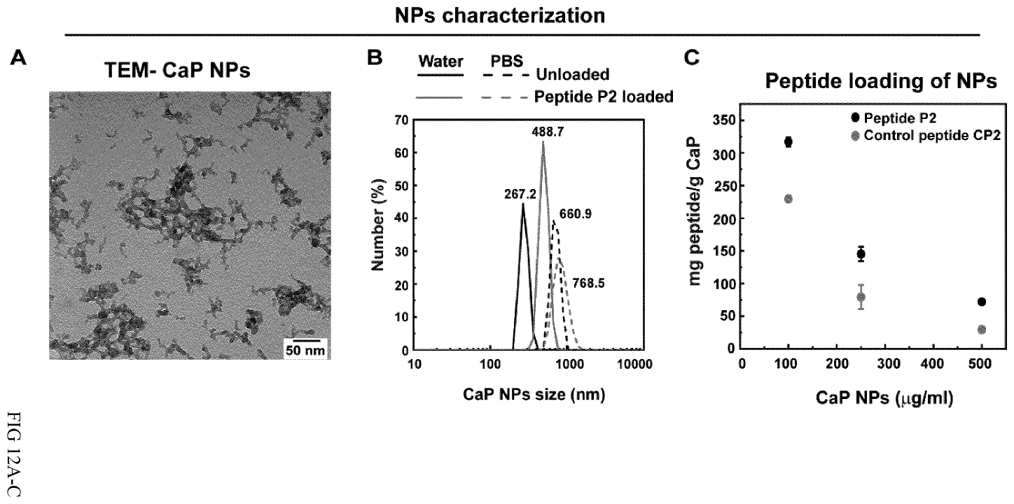

The inventors found that intracellular T4R bacteria in DCs co-localized with MRC-1 but did not co-stain with the autophagy marker LC3B (FIGS. 4F and 11A). This was in contrast to T4RΔply which co-localized with LC3B (FIGS. 4G and 11A). In DCs treated with peptide P2, intracellular T4R did not co-localize with MRC-1, but co-stained with LC3B, suggesting that the bacteria are targeted for degradation by autophagy. In contrast to P2, treatment with the control peptide CP2 did not promote co-localization of intracellular bacteria with LC3B. The inventors also examined the intracellular fate of SLO-producing S. pyogenes type M1T1, and its isogenic SLO mutant. The results were consistent with those for S. pneumoniae, showing that the SLO-producing strain co-localized with MRC-1, but not with LC3B. Upon addition of peptide P2, intracellular bacteria co-stained with LC3B (FIG. 11B, C). The SLO mutant strain consistently co-stained with LC3B, irrespective of peptide treatment.

### Example 6: Treatment with Peptide P2 Reduces Development of Pneumococcal Disease In Vivo

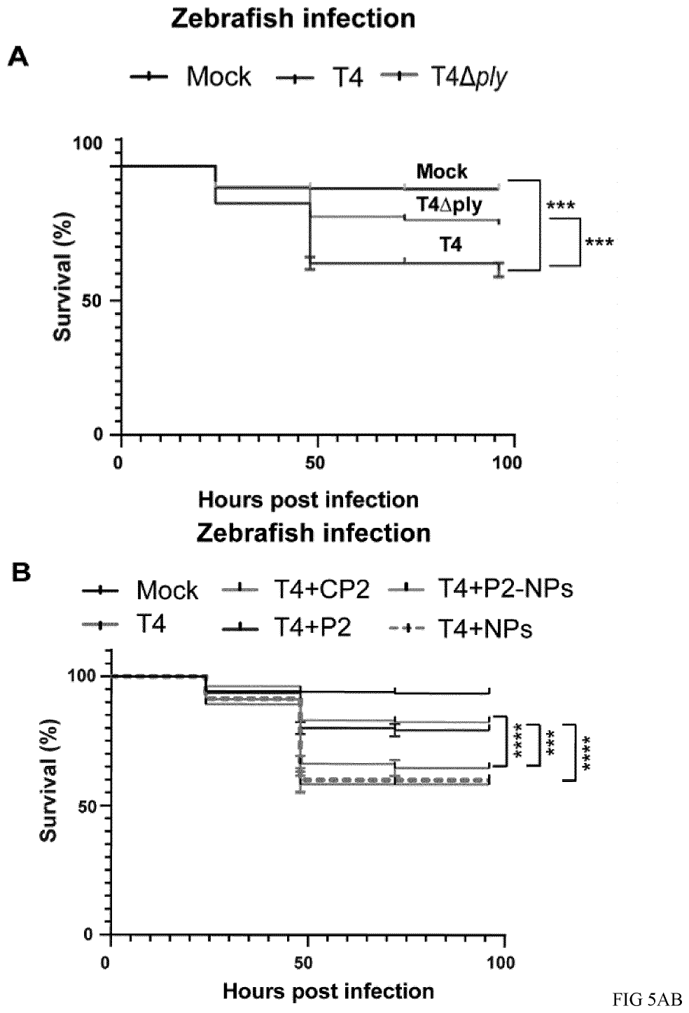

To assess the therapeutic potential of peptide P2 against pneumococcal infection in vivo, the inventors first used a zebrafish (Danio renio) embryo model in which pneumococci were microinjected into the yolk sac of fertilized embryos. Zebrafishes have been shown to be useful models to study infectious diseases owing to their optical transparency, ease of high-throughput screening, and conservation of the major components of the human immune system. Also, they have been used to study pneumococcal pathogenesis. Thus, the inventors infected zebrafish embryos 3-4 hours post fertilization with 500 CFU of wild-type T4 or its isogenic PLY mutant, T4Δply. The inventors found that embryos infected with T4Δply showed significantly higher survival in comparison to the wild-type T4 strain (FIG. 5A) confirming the importance of PLY in pneumococcal virulence. Importantly, co-administration of peptide P2 (1 nM) during T4 infection significantly improved the survival compared to infection alone (FIG. 5B). In contrast, the control peptide CP2, that does not bind PLY, did not show any significant effect on the survival of infected zebrafishes, confirming the specificity of peptide P2.

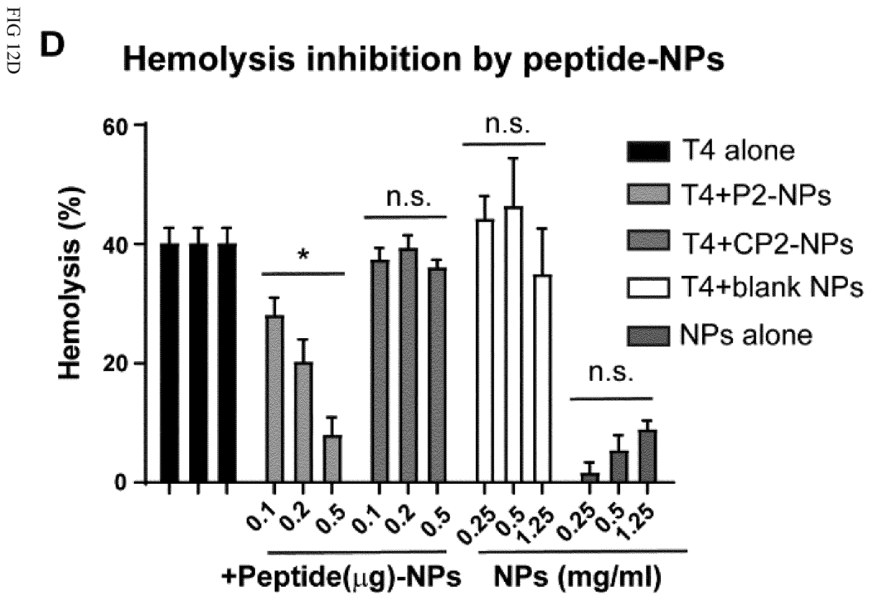

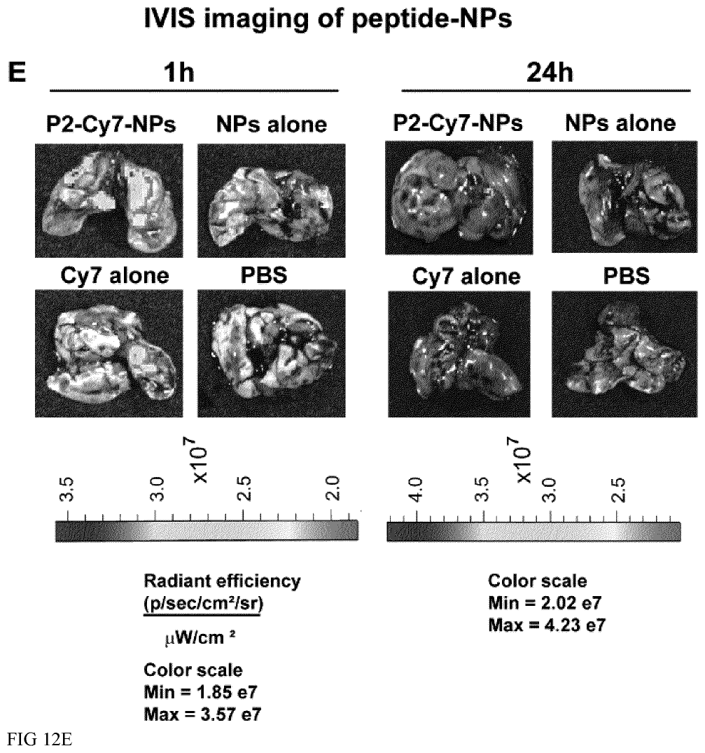

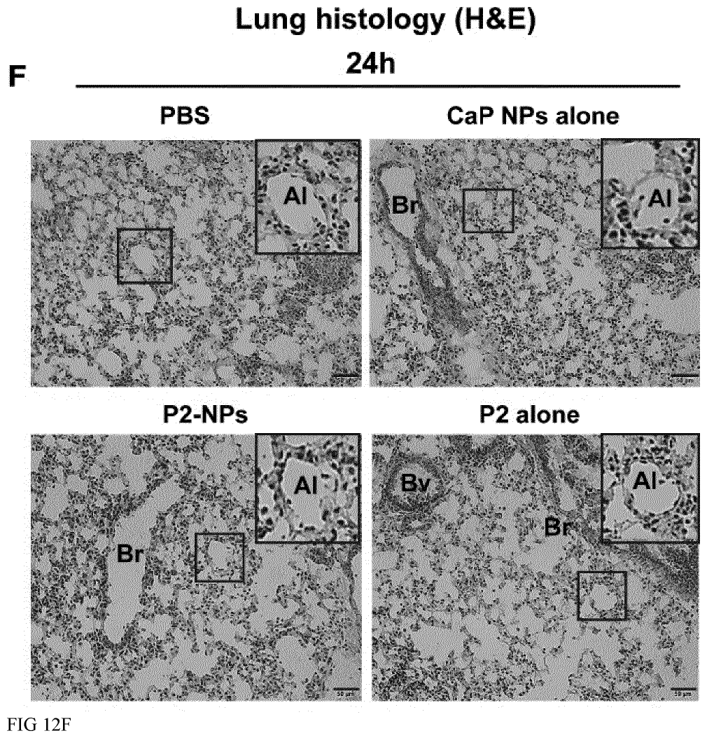

Although peptides are specific, they have a limited stability and bioavailability in vivo. Therefore, the inventors used biocompatible calcium phosphate (CaP) nanoparticles (NPs) as peptide nanocarriers to minimize degradation and to achieve rapid targeting to the lungs. Peptide conjugation to nanoparticles also allows for their non-invasive delivery by inhalation for rapid targeting to the lungs rather than the conventional intravenous route. Thus, the MRC-1 peptide, P2 and control peptide, CP2 were loaded onto CaP NPs by physisorption upon overnight incubation as described previously. The morphology and size distribution of the NPs for peptide loading were characterized using transmission electron microscopy (FIG. 12A) and dynamic light scattering (FIG. 12B). The particles exhibited the characteristic fractal-like agglomerate nanostructure (Sauter mean diameter ˜8 nm) and the mean hydrodynamic size of the peptide-loaded NPs was ˜770 nm when suspended in PBS. The amount of peptide loaded onto CaP NPs was quantified using the BCA assay (FIG. 12C). Up to 75-150 mg peptide/g CaP could be loaded at a particle concentration of 250 μg/ml. Further, the peptide activity upon conjugation to NPs was verified by hemolysis assay and a dose-dependent inhibition of T4-induced hemolysis by peptide P2 loaded nanoparticles (P2-NPs) was observed, but not by nanoparticles conjugated with control peptide CP2 (CP2-NPs) (FIG. 12D). The unloaded NPs alone did not induce any significant hemolysis (FIG. 12D). Importantly, the inventors found that co-injection of zebrafish embryos with P2-loaded NPs (1 nM) and strain T4 improved survival of the fishes compared to unloaded NPs and T4 alone (FIG. 5B).

Next, the inventors used an intranasal mouse pneumonia model to investigate the effects of the peptide-loaded NPs on bacterial virulence in vivo. First, the inventors studied lung delivery and in vivo distribution of the peptide nanocarriers by administering Cy7-tagged P2-CaP NPs intranasally to mice and imaging the lungs post-mortem using the IVIS imaging at 1 h and 24 h post administration. Unloaded NPs and Cy7 dye alone were used as negative and positive controls, respectively. Images showed that P2-NPs were efficiently distributed in both lung lobes at 1 h and the peptide could be detected in the lungs even at 24 h (FIG. 12E). Peptide P2 or P2-NPs were not toxic as revealed by the lung histology analysis, which showed that the mice had a normal lung morphology with intact alveolar space and absence of inflammatory cells (FIG. 12F). Moreover, these mice did not develop any clinical symptoms at 24 h post administration. Subsequently, the inventors challenged mice intranasally with 2×10⁶ CFUs of S. pneumoniae T4 strain or the PLY mutant, T4Δply, combined with peptide P2 (5 μg/mouse) or peptide conjugated nanoparticles (5 μg peptide; 25 μg CaP NPs/mouse) in 50 μl PBS. In agreement with the zebrafish data, mice infected with T4Δply showed higher survival compared to T4 infected mice (FIG. 5C). Further, mice treated with peptide P2 showed prolonged survival in comparison to the infected group. Indeed, peptide P2 treated mice had similar survival as T4Δply infected mice, indicating that the peptide effectively inhibited PLY in vivo. Importantly, administration of peptide P2 conjugated NPs resulted in a significantly higher survival (50%) at the end of the experiment in comparison to mice treated with blank NPs or only infected (FIG. 5C). Also, P2-NPs treated mice showed higher survival in comparison to the free peptide indicating that the NPs improved the peptide efficacy in vivo. In accordance with the higher survival, P2-NPs treated mice also had significantly reduced bacterial load in the lungs compared to mice infected only or challenged with blank NPs (FIG. 5D). In addition, treatment with peptide P2 or P2-NPs significantly reduced the levels of inflammatory cytokines, TNF-α (FIG. 5E) and IL-12 (FIG. 5F) in the lung tissue.

### MATERIALS AND METHODS

### Study Design

The main objective was to construct peptides from the human mannose receptor, MRC-1, as decoys to inhibit bacterial CDCs and treat pneumococcal infection. The study design consisted of (i) computational modelling of interaction between MRC-1 and bacterial CDCs and designing peptides from region of interaction, (ii) in vitro cell culture and 3D lung tissue models to screen peptide efficacy, (iii) synthesis of peptide conjugated nanoparticles for intranasal delivery of peptides and (iv) in vivo validation of therapeutic peptide nanocarriers using zebrafish and mice infection models. First, the inventors used the zebrafish (Danio renio) embryo model to assess the protection conferred by peptides against pneumococcal infection. To rule out non-specific effects of the peptide, a control peptide which does not bind the bacterial toxin was included in the zebrafish study. The zebrafish embryos were randomly assigned to the treatment groups and a minimum of 156 embryos/group were used. All experiments were performed thrice. The zebrafish study was approved by the ethical review board, Stockholm Animal Research Committee (Drn 19204-2017) and the Swedish Board of Agriculture. Next, the inventors used the mouse model to validate the efficacy of peptide nanocarriers using an intranasal infection model. 6 weeks old male C57BL/6 wild-type mice were used (Charles River, Germany). The mouse experiments were randomized and included 10 mice per treatment group. The sample size was determined based on previous experience and accounting the mortality rate. Appropriate controls were included to exclude unspecific effects of the peptide or nanoparticles alone. The mouse experiments were approved by the local ethical committee (Stockholms Norra djurförsöksetiska nämnd).

### Bacterial Strains and Growth Conditions

The encapsulated S. pneumoniae serotype 4 strain TIGR4 (T4; ATCC BAA-334), and the type 2 strain D39 were used in this study, as well as the isogenic capsule and pneumolysin deletion mutants, T4R (J. Fernebro et al., Capsular expression in Streptococcus pneumoniae negatively affects spontaneous and antibiotic-induced lysis and contributes to antibiotic tolerance. The Journal of infectious diseases 189, 328-338 (2004)) and T4RΔply (M. Littmann et al., Streptococcus pneumoniae evades human dendritic cell surveillance by pneumolysin expression. EMBO molecular medicine 1, 211-222 (2009)), respectively. Pneumococci were grown on blood agar plates overnight and colonies were inoculated into pre-warmed C+Y medium and grown to OD=0.4 at 37° C. The Streptococcus pyogenes strain 5548 of serotype M1T1and the isogenic SLO mutant were obtained from Prof. Victor Nizet, University of California, San Diego. Streptococcus pyogenes was grown on brain heart infusion agar plates overnight and inoculated into Todd Hewitt Broth containing 0.5% yeast extract at 37° C. Listeria monocytogenes type 1 strain (ATCC 19111) was obtained from the Swedish Institute for Infectious Disease Control, and grown on brain heart infusion agar plates overnight and inoculated into on brain heart infusion broth at 37° C.

### Purified Bacterial Toxins and MRC-1 Peptides

Recombinant toxins, PLY, LLO and SLO, were expressed and purified at the Protein Production Platform, Nanyang Technological University, Singapore. The purified endotoxin free toxoid derivative, Pdb PLY (W433F), was a gift from Prof. Aras Kadioglu, University of Liverpool, UK. The purified LLO toxoid derivative, LLO (W489F) was a gift from Prof. Gregor Anderluh, National Institute of Chemistry, Slovenia. The overlapping 13-mer MRC-1 peptides (described in Table S1) were commercially synthesized and purified to >95% purity by Genscript (NJ, USA). The lyophilized peptides were dissolved in ultrapure water or analytical grade dimethyl sulfoxide (Sigma) according to the manufacturer's suggestions.

### Mouse Infection Model

All animal experiments were approved by the local ethical committee (Stockholms Norra djurförsöksetiska nämnd). 6 weeks old male C57BL/6 wild-type mice were used (Charles River, Germany). The study included 10 mice/individual group which were randomly assigned to the different treatment groups. Sample size calculations were determined according to previous experience. Mice were anesthetized by inhalation of 4% isofluorane (Abbott) mixed with oxygen, and intranasally administered with 50 μl of PBS containing 2×10⁶ CFU of S. pneumoniae T4, or the PLY mutant T4Δply, or T4 mixed with the peptide P2 alone (5 μg/mouse), or P2-conjugated to CaP NPs (5 μg peptide; 25 μg CaP NPs/mouse) or blank NPs (25 μg/mouse). Clinical symptoms of mice were monitored for three days post infection and sacrificed upon reaching humane end points according to ethical regulations. The mice were checked twice every day; morning at 9 am (referred as “early check”) and evening at 7 pm (referred as “late check”). Post-mortem, lungs were collected and washed in PBS and homogenized by passing through the 100 μm cell strainer. Bacterial counts were determined from lung homogenates by viable count on blood agar plates. Aliquots of lung homogenates were frozen at −80 ° C. for cytokine quantification by ELISA.

### IVIS Imaging to Visualize Biodistribution of Peptide Conjugated NPs in Mice

To verify the delivery of peptide-conjugated NPs to the lungs, mice were anesthetized by inhalation of 4% isofluorane mixed with oxygen and administered intranasally (50 μl/mouse) with Cy7 conjugated peptide P2-NPs (5 μg peptide; 25 μg CaP NPs/mouse) or non-fluorescent NPs alone (negative control), or Cy7 dye alone (positive control) or PBS. Mice were sacrificed either at 1 h or 24 h post treatment. Lungs were collected post-mortem. Cy7 fluorescence in the mouse lungs was imaged using the IVIS Spectrum-CT Imaging system (Caliper-Perkin Elmer). RGB Profile Plot representing the signal intensity was generated for each image taken with the IVIS Spectrum System.

### Zebrafish Infection Model

The zebrafish study was approved by the ethical review board, Stockholm Animal Research Committee (Drn 19204-2017) and the Swedish Board of Agriculture. The AB-strain of zebrafish embryos (Danio renio) was collected within first hours post fertilization (hpf) from the zebrafish core facility at Karolinska Institutet, Stockholm and maintained at 28.5° C. in E3 medium (5.0 mM NaCl, 0.17 mM KCl, 0.33 mM CaCl₂, 0.33 mM MgSO₄). The fertilized embryos (3-4 hpf) were microinjected into the yolk sac with 1 nL of E3 medium containing 500 CFU of S. pneumoniae T4 alone or T4 mixed with peptide P2 or control peptide CP2 (1 nM) or T4 mixed with P2-conjugated NPs or blank NPs. Microinjection was done using a glass needle (Harvard apparatus, Quebec, Canada) controlled with a micromanipulator Narishige MN-153 (Narishige International Limited, London, UK) connected to an Eppendorf FemtoJet express (Eppendorf AG, Hamburg, Germany). The injected volume was previously optimized by injecting of a droplet into mineral oil over a scale bar. The embryos were randomly assigned to the treatment groups and a minimum of 156 embryos/group was used. To determine the actual number of bacteria in the injected volume, one drop was collected into the agar plates, and 1-3 embryos were digested and plated just immediately after injection. The infected embryos were incubated at 30° C. for 96 hours. The infected embryos were monitored for disease symptoms and survival under a stereomicroscope twice a day up to four days post injection. Absence of heartbeat and movement was interpreted as a sign of death. All experiments were performed thrice.

### Primary Monocyte-Derived Dendritic Cells, Cell Culture and Infection

Human DCs were differentiated from primary monocytes isolated from anonymous buffy coats of healthy blood donors (Karolinska University Hospital). The monocytes were isolated by using the RosetteSep monocyte purification kit (Stem Cell Technologies). For differentiation into DCs, monocytes were cultured in R10 (RPMI 1640, 2 mM L-glutamine, 10% FBS) supplemented with GM-CSF (40 ng/ml) and IL-4 (40 ng/ml) from Peprotech for 6 days. DCs were verified by flow cytometry to be >90% CD1a⁺CD11c⁺. For infection, DCs were incubated with bacteria at MOI of 10 and the extracellular bacteria were killed using gentamicin (200 μg/ml) after 2 h post infection.

Human monocytic leukemia THP-1 cells (ATCC TIB-202) were cultivated in R10 medium and maintained at density of 10⁶ cells/ml. For differentiation into macrophages, THP-1 cells were treated for 48 h with 20 ng/ml of phorbol myristate acetate (Sigma). For cytokine measurements, differentiated THP-1 macrophages were incubated with purified PLY, LLO and SLO (0.5 μg/ml) in the presence of 100 μM peptides P2, P3 and control peptide, CP2 and the culture supernatants were collected 18 h later for cytokine ELISA.

### 3D Lung Epithelial Tissue Model

The organotypic lung epithelial tissue model was set up as previously described (A. T. Nguyen Hoang et al., Dendritic cell functional properties in a three-dimensional tissue model of human lung mucosa. American journal of physiology. Lung cellular and molecular physiology 302, L226-237 (2012)). The human lung fibroblast cell line, MRC-5 (ATCC, Manassas, VA), was cultured to between 80-90% confluence and maintained in Dulbecco's Modified Eagle's Medium (DMEM) (GE Healthcare Life Sciences, Marlborough, MA,) supplemented with 10% heat-inactivated fetal bovine serum (FBS) (Sigma Aldrich, St. Louis, MO), 2 mM L-glutamine (GE), 1 mM sodium pyruvate (GE), and 10 mM HEPES buffer (GE). The human lung epithelial cell line 16HBE14o- (gift from Dr. Dieter Gruenert, Mt. Zion Cancer Center, University of California, San Francisco, CA) was modified to express GFP (A. T. Nguyen Hoang et al., Technical advance: live-imaging analysis of human dendritic cell migrating behavior under the influence of immune-stimulating reagents in an organotypic model of lung. J Leukoc Biol 96, 481-489 (2014)) and cultured in a tissue-culture flask coated with fibronectin solution containing 1 mg/ml bovine serum albumin (0.1%), 3 mg/ml bovine collagen type I (Advanced BioMatrix, San Diego, CA), and 1 mg/ml human fibronectin (Corning Inc., Corning, NY) in PBS. GFP-expressing 16HBE14o- (henceforth, GFP-16HBE) were grown to between 80-90% confluence in Minimum Essential Medium (MEM) (Thermo Fisher Scientific, Waltham, MA, Gibco) containing 10% FBS, 10 mM HEPES buffer, 2 mM L-glutamine, and 1× nonessential amino acids (Gibco).

The model was prepared by first seeding an acellular collagen layer (1 ml) in 6 well plate Transwell inserts (Corning Inc.) and then allowing the layer to gel for 30 minutes at 37° C. and 5% CO₂. After gelation, a cellular layer (3 ml) containing MRC-5 cells suspended in a collagen matrix was added to the model and allowed to gel for 2 h prior to the addition of complete DMEM. Media was changed the next day and subsequently every second day for 6-7 days until the MRC-5 cells were fully embedded in the collagen matrix. Once ready, the apical media was removed and 50 μl of GFP-16HBE cells were added to the apical side of the models at a density of 1.6×10⁶cells/ml (80,000 cells/model), allowed to settle and adhere for 2 h, and then the complete model was submerged in complete DMEM for 3-4 days until confluent. Once confluent, models were airlifted and maintained at an air-liquid interface (ALI) for a minimum of 5 days prior to stimulation. The acellular collagen layer solution was composed of complete DMEM, 3 mg/ml bovine collagen type I, and a premix solution containing 5× DMEM, 2 mM L-glutamine, 71.2 mg/ml NaHCO₃, 45% FCS, and 50 mg/mL gentamicin (Sigma Aldrich). The cellular layer contained the premix solution, 3 mg/ml bovine collagen type I, complete DMEM, and MRC-5 cells at a density of 2.3×10⁵cells/ml (75,000 cells/model). Models were maintained in complete DMEM for the entirety of the experiment.

### Hemolysis Assay

Human blood from anonymous healthy donors (obtained from Karolinska University Hospital) was diluted 1:100 in PBS with 0.5 mM DTT and mixed 1:1 with two-fold serial dilutions of 108 CFUs of bacteria or 1 μg/ml purified bacterial toxins in 96 well plates. The MRC-1 peptides were serially diluted ten-fold (1-1000 μM) in PBS and added to the wells prior to addition of blood. The blood was co-incubated with whole bacteria or purified toxins at 37° C. for 1 h and after 50 minutes 0.1% triton X-100 was added to the positive control wells. Cells were spun down at 400 g for 15 min and the absorbance of the supernatants was measured at 540 nm in a microplate reader. Percentage of lysis compared to the positive control was calculated. All samples were assayed in triplicates.

### ELISA to Test Binding of Peptides to PLY, LLO and SLO

Briefly, 96-well flat-bottomed plates (Sigma, UK) were coated overnight with 10 μM of MRC-1 peptides in coating buffer (15 mM Na₂CO3, 35 mM NaHCO₃, pH 9.6). Wells were blocked with 200 μl of 10% (v/v) FBS in PBS for 2 h, and then washed three times with PBS, 0.05% (v/v) Tween 20 (Sigma). 50 μl of purified PLY, LLO and SLO (0-1 μM) in PBS was added and incubated at 37° C. for 1 h. 1 μM BSA was added to control wells. Wells were washed with PBS and bound proteins were detected by adding 100 μl of mouse α-PLY (1:1000), mouse α-SLO (1:400), rabbit α-LLO (1:1500) (Abcam) respectively in blocking buffer. Plates were incubated with 100 μl of secondary anti-rabbit/anti-mouse IgG HRP (1:2000) dilution in blocking buffer. Bound antibodies were detected by adding 100 μl of tetramethylbenzidine substrate solution for 30 min. Phosphoric acid (1 M) was used as the stop solution and absorbance was measured at 450 nm.

### LDH Cytotoxicity Assay

Cytotoxicity was determined by measuring the activity of lactate dehydrogenase enzyme released into the culture supernatant using the Cytotoxicity Detection kit (Roche) according to the manufacturer's instructions. The percentage cytotoxicity was calculated by ratio to 100% lysis control (0.1% saponin).

### Cytokine ELISA

For cytokine assays, the cell-free culture supernatants were collected 18 h post stimulation and frozen at −20° C. The levels of TNF-α, IL-8 and IL-12(p70) in the culture supernatants and mouse lung tissue were analyzed using the OptElA ELISA kit (BD Biosciences) following the manufacturer's instructions.

### Co-Localization of MRC-1 with Purified PLY, LLO and SLO in DCs

For co-localization studies with purified toxins, human DCs (3x105) were attached on poly-lysine coated coverslips and incubated with 0.2 μg/ml of purified PLY, LLO and SLO for 45 min at 37° C. and washed twice with PBS. The toxoid derivatives, PLY(W433F) and LLO (W489F), were used as negative controls. The cells were fixed with PBS buffered 4% paraformaldehyde for 10 min. The cells were permeabilized with 0.5 for 15 min in dark. Non-specific interactions were blocked by incubating cells with 5% FBS in PBS for 30 min. The cells were then incubated overnight with Alexa 647-conjugated rabbit anti-EEA-1 (Abcam) to stain early endosomes. PLY and SLO was detected using mouse anti-PLY (Abcam), or mouse anti-SLO (Abcam) followed by secondary goat anti-mouse secondary antibody (Thermo Fisher Scientific). LLO was detected using rabbit anti-LLO conjugated to Alexa 488 using the Zenon Rabbit IgG Labeling kit (Abcam). MRC-1 was detected using Alexa 594-conjugated-Rabbit anti-MRC1 (Abcam). The coverslips were mounted on slides using Prolong Gold anti-fade mounting medium containing the nuclear stain 4,6-diamidino-2-phenylindole (DAPI; Thermo Fisher Scientific). Images were acquired using a Delta Vision

Elite microscope under the 100× oil immersion objective (GE Healthcare).

### Computational Docking of MRC-1 CTLD4 with Bacterial Toxins

Computational docking of the CTLD4 domain of MRC-1 with the pore-forming toxins PLY, LLO and SLO was performed using the ClusPro 2.0 docking server (D. Kozakov et al., The ClusPro web server for protein-protein docking. Nature protocols 12, 255-278 (2017)). The available crystal structures of MRC-1 CTLD4 (PDB id-1EGG), PLY (PDB id-5CR6), LLO (PDB id-4CDB) and SLO (PDB id-4HSC) were obtained from the Protein Data Bank. Docking was performed using the balanced model, considering electrostatic and hydrophobic interactions. The models were ranked based on the size of the cluster that is defined as the ligand position with the most neighbors within 9 A distance. The docking models were analyzed using the PyMOL Molecular graphics software version 2.0.6. The receptor (MRC-1) was colored red and the ligands PLY, LLO and SLO were colored green. The eukaryotic membrane binding undecapapetide loop in domain 4 of PLY, LLO and SLO was labelled yellow. The zoomed structures show the precise amino acid residues in MRC-1 (red) and the undecapeptide loop of PLY, LLO and SLO (yellow) that are predicted to interact with each other.

### CFU Plating Assay to Quantify Intracellular Bacteria in DCs

Briefly, DCs were infected with pneumococci, type 2 (D39) or type 4 (T4) bacteria at MOI of with or without adding 100 μM of the MRC-1 peptides, P2 or CP2. At 2 h post infection, gentamicin (200 μg/ml) was added and incubated for 1 h at 37° C. to kill extracellular bacteria. The anti-PLY antibody (1:100) was used as control to ascertain the role of PLY in bacterial invasion into DCs. The actin polymerization inhibitor, cytochalasin D (0.5 mM), was used as control to inhibit bacterial uptake by DCs. The cells were washed, resuspended in 100 μl PBS, and serial dilutions were plated on blood agar plates in 10 μl volume and incubated overnight.

### Statistical Analysis

Data were statistically analyzed using GraphPad Prism v.5.04. Data represent mean±s.e.m. For comparison between groups, the one way or two way-ANOVA test with Bonferroni or Dunnett's post test for multiple comparisons was used. Comparison of survival curves in mice and zebrafish was performed using the Log-rank (Mantel-Cox) test. Normalized data were analyzed using paired t-tests. Differences were considered significant at *P<0.05, **P<0.01; ***P<0.001, ****P<0.0001. n.s. denotes that the difference is not significant.

### Nanoparticle Synthesis and Characterization

Calcium phosphate (CaP) NPs (dBET =8 nm) were produced by flame spray pyrolysis as described previously (1). The metal-organic precursors calcium acetate hydrate 99%, Sigma-Aldrich) and europium nitrate hexahydrate (99.9%, Alfa Aesar) were dissolved in a mixture of 2-ethylhexanoic acid (99%, Sigma-Aldrich) and propionic acid 99.5%, Sigma-Aldrich) in 1:1 ratio and stirred under reflux for 30 min at 70° C. The nanoparticles were doped with Europium to enable their monitoring by luminescence. Subsequently, tributyl phosphate (≥99%, Sigma-Aldrich) was added, after a clear solution was observed, in appropriate quantity in order to obtain Ca/P molar ratio of 2.19. The total metal concentration of the precursor solution was 0.1 M. The precursor solution was fed to the FSP nozzle through a capillary tube (SGE Analytical Science) using a syringe pump (New Era Pump Systems, Inc.). The solution was atomized in the FSP nozzle by oxygen gas at 3 L/min (Strandmöllen AB) (EL-FLOW Select, Bronkhorst) at constant pressure drop (1.8 bar). The synthesis of the particles was carried out at 8 ml/min precursor feed flow rate. The spray flame was ignited by a premixed supporting flame of methane/oxygen (Scientific grade, Linde Gas AB) at flow rates of 1.5 L/min and 3.2 L/min, respectively. The particles were collected on a glass fiber filter (Hahnemühle) with the aid of a Mink MM 1144 BV vacuum pump (Busch).

The specific surface area (SSA) was determined by the nitrogen adsorption-desorption isotherms in liquid nitrogen at 77K using a Tristar II Plus (Micromeritics) instrument. The sample was degassed for at least 3 h at 110° C.

The structure of the NPs was observed using transmission electron microscopy (TEM) in a FEI Tecnai BioTWIN instrument operated with an acceleration voltage of 120 kV and equipped with a 2k×2k Veleta OSiS CCD camera. For the TEM imaging, the nanoparticles were suspended in ethanol in a water-cooled cup horn system (VCX750, cup horn Part no. 630-0431, Sonics Vibracell) (10 min, 100% amplitude) and one drop of the suspension was deposited onto a carbon coated copper grid (400 mesh carbon film, S160-4, Agar Scientific). The grid was dried at ambient temperature overnight.

Size distribution of unloaded and MRC1-peptide loaded CaP NPs was evaluated by dynamic light scattering (DLS) with a zetasizer ultra (Malvern Panalytical).

### MRC1-Peptide Adsorption Onto CaP NPs

The MRC1 peptide was loaded onto CaP NPs via physisorption (2). Suspensions of MRC1-loaded CaP NPs in PBS pH 7.4 (200 μl sample volume) were prepared by addition of 100 μl of dispersed CaP NPs in PBS pH 7.4 of initial concentration ranging from 200 to 1000 μg/ml to an equal volume of MRC1 peptide solution (initial concentration of 200 μg/ml). The suspensions were placed on a roller mixer (Stuart SRT9D) for gentle mixing at 60 rpm overnight. The particles were separated via centrifugation at 10000 rpm for 20 min and the supernatant containing the unloaded peptide was collected for quantification using a Pierce bicinchoninic acid (BCA) protein assay kit (Thermo Fisher Scientific) according to manufacturer's instructions. Absorbance was measured at 562 nm using a microplate reader (SpectraMax Plus, Molecular Devices) and the amount of peptide was calculated from a calibration curve. The amount of loaded peptide was calculated from the difference between the initial concentration and the concentration of the supernatant. Furthermore, the loaded particles were washed once with PBS and re-dispersed in PBS. The amount of peptide after the washing was also quantified in the supernatant after centrifugation (10000 rpm, 20 min) and was found negligible 1%) indicating the stability of the conjugates. The final concentration of peptide P2 on the CaP NPs varied between 75-150 mg/g CaP at a NP concentration of 250 μg/ml.

For in vivo imaging of the peptide conjugated NPs, the inventors generated fluorophore-loaded peptide NPs by incorporating the near infrared dye, Sulfo-Cy7 amine (Lumiprobe, GmbH) as described previously (3). An aqueous solution of the Cy7 (initial concentration 62.5 μg/ml) was co-incubated with MRC-1 peptide loaded NPs overnight on a roller shaker at 60 rpm (Stuart SRT9D). The unconjugated dye was removed by at least 3 washings and centrifugation at 10000 for 15 min. The amount of Cy7 loaded on CaP nanoparticles was measured using an ultraviolet-visible (UV-Vis) spectrophotometer (NanoDrop One, Thermo Scientific) (λ=750 nm). The concentration of Cy7 was calculated as the difference between the concentration of the initial solution and that of the supernatant. The final concentration of Cy7 within the loaded CaP particles was 29.2±2.24 μg/ml.

### Live Imaging of Cytolysis by Bacterial Toxins

Human THP-1 monocytes were seeded at 5×10⁵ cells in 12 well plates and differentiated with PMA (20 ng/ml) for 48 h in 12 well plates. Cells were washed with PBS and loaded with live/dead reagent (2 μM Calcein AM and 4 μM Ethidium bromide) for 20 min at 37° C. The cell-permeable dye, Calcein AM, becomes green-fluorescent upon hydrolysis by intracellular esterases in live cells, while dead cells are stained red by propidium iodide. Then, 0.5 μg/ml PLY, LLO or SLO with or without 100 μM peptide P2 or control peptide, CP2, was added to the wells. Cholesterol (100 μM) was used as a positive control, while BSA (100 μM) was used as negative control. The plate was mounted on the microscope stage set at 37° C. and 5% CO₂ and imaged at 30 second interval for total time of 20 min under the green (488 nm emission) and red (594 nm emission) channels; imaging was performed every 30 seconds to avoid cytolysis induced by the cytotoxic effect of the excitation laser. Images were acquired using a Delta Vision Elite microscope under a 20X objective (GE Healthcare).

### Live Imaging of the 3D Lung Model

After 5 days of air exposure, models were cut out from the Transwell inserts and mounted for live imaging. For mounting, 50 μl of media containing stimulation samples were added to the base of a glass-bottomed well plate (MatTek Corp., Ashland, MA) followed by placement of the separated models apical-side down into the 50 μl to ensure exposure of the model to the sample. A 4% (w/v) low-temp gelling agarose solution was then added around the top of the inverted model (basolateral side) and 1 ml of complete DMEM was added around the outside of the agarose ring to provide nutrients and maintain humidity during the imaging. Once mounted, models were maintained at 37° C. and 5% CO₂ and imaged at 5 min intervals starting from 45 min post stimulation until 240 min post stimulation; maximum intensity projections were created for each time point using the Nikon NIS Elements Software (Nikon Inc., Tokyo, Japan), and total GFP expression was then analyzed from each frame over time. Statistical analyses were conducted using Prism (GraphPad Software, San Diego, CA). All images were obtained on a Nikon A1R HD25 confocal microscope at 20X magnification.

### Co-Localization of Bacteria with MRC-1 and LC3B in DCs

Briefly, 2×10⁵ DCs seeded onto coverslips were infected with the unencapsulated type 4 S. pneumoniae T4R and its isogenic PLY mutant, T4RΔply, or the S. pyogenes strains M1T1 strain M1T1 and its isogenic SLO mutant at MOI of 10. The MRC-1 peptides, P2 and CP2, were diluted in R10 medium and used at 100 μM. At 2 h post infection, extracellular bacteria were killed by adding gentamicin (200 μg/ml) for 60 min and washed twice with PBS. The DCs were fixed, permeabilized and blocked as described earlier and stained with 1:1000 diluted Alexa 647-conjugated rabbit anti-LC3B antibody (Abcam) overnight. Pneumococci were detected using 1:100 diluted rabbit anti-pneumococcal anti-serum (Eurogentec) labelled with Alexa 488 using a Zenon Rabbit IgG Labeling kit (Thermo Fisher Scientific) for 1 h at room temperature. Streptococcus pyogenes (Group A streptococci) was detected using 1:50 diluted Alexa 488 conjugated rabbit anti S. pyogenes (Abcam) overnight at 4° C. MRC-1 was detected using Alexa 594-conjugated-Rabbit anti-MRC1 (Abcam). The coverslips were mounted on slides using Prolong Gold anti-fade mounting medium containing the nuclear stain 4,6-diamidino-2-phenylindole (DAPI; Thermo Fisher Scientific). Images were acquired using a Delta Vision Elite microscope under the 100× oil immersion objective (GE Healthcare). The cell boundary was marked by the DC receptor, MRC-1. In some images, LC3B was pseudo-colored to cyan for better color contrast, for quantification of percentage of intracellular bacteria that co-localized with MRC-1 and LC3B.

1. A method of treatment or prevention of a bacterial infection by a pathogen expressing a cholesterol-dependent cytolysin (CDC), comprising administering to a subject in need thereof a polypeptide or peptidomimetic comprising:
a sequence of at least 7 residues differing by residue substitutions, deletions or insertions numbering 0-2 compared to the sequence:
(SEQ ID NO: 1)
GLTYGSPSEGFTWSDGSPVSYENWAYGEPNNYQNVEYCGELKGDPTMSW
NDINCEHLNNWICQ,
wherein the sequence comprises YEN residues aligning with the bolded sequence at positions 21-23 of SEQ ID NO: 1; and
wherein the polypeptide or peptidomimetic comprises no more than 100 residues in total.

2-3. (canceled)

4. The method according to claim 1, wherein the polypeptide or peptidomimetic comprises no more than 50 residues in total.

5. The method according to claim 1, wherein the sequence comprises a T residue aligning with the position 12 of SEQ ID NO: 1 and a S residue aligning with the position 14 of SEQ ID NO: 1.

6. (canceled)

7. The method according to claim 1, wherein the polypeptide or peptidomimetic comprises the sequence VSYENWA (SEQ ID NO: 4).

8-9. (canceled)

10. The method according to claim 1, wherein the subject is in need of treatment or prevention of pneumococcal disease.

11. The method according to claim 10, wherein the subject is in need of treatment or prevention of a pneumococcal disease selected from pneumococcal sinusitis, pneumococcal otitis, pneumococcal pneumonia and invasive pneumococcal disease including but not limited to pneumococcal sepsis and pneumococcal meningitis, preferably invasive pneumococcal disease.

12. The method according to claim 1, wherein the subject is in need of prevention of streptococcal carriage, such as carriage of Group A streptococci such as S. pyogenes.

13. The method according to claim 1, wherein the subject is in need of treatment or prevention of secondary bacterial pneumonia following a virus infection.

14. The method according to claim 1, wherein the subject is in need of treatment or prevention of listeriosis.

15. The method according to claim 1, wherein the subject is in need of treatment or prevention of necrotizing fasciitis, skin infections or septicaemia.

16. The method according to claim 15, wherein the disease treated or prevented is caused by Group A streptococci such as S. pyogenes.

17. The method according to claim 1, wherein the administration involves intranasal administration of the polypeptide or peptidomimetic to a subject in need thereof.

18. (canceled)

19. The method according to claim 1, wherein the polypeptide or peptidomimetic comprises the sequence FTWSDGSPVSYEN (SEQ ID NO: 2), or a subsequence thereof being at least 5 residues long.

20. The method according to claim 1, wherein the polypeptide or peptidomimetic comprises YENWAYGEPNNYQ(SEQ ID NO: 3), or a subsequence thereof being at least 5 residues long.

21. The method according to claim 1, wherein the polypeptide or peptidomimetic comprises a consecutive sequence of at least 13 residues differing by residue substitutions, deletions or insertions numbering 0-2 compared to the sequence SEQ ID NO: 1.

22. The method according to claim 1, wherein the polypeptide or peptidomimetic comprises a consecutive sequence of no more than 60 residues having more than 80% sequence identity to SEQ ID NO: 1.

23-24. (canceled)

25. The method according to claim 1, wherein the residue substitutions, deletions or insertions number 0 in total.

26-33. (canceled)

34. The method according to claim 1, wherein the polypeptide or peptidomimetic competitively interferes with the cholesterol-binding activity of pneumolysin (PLY), streptolysin (SLO) and/or listeriolysin (LLO), preferably PLY.

35. (canceled)

36. A composition, comprising a polypeptide or peptidomimetic comprising:
a sequence of at least 7 residues differing by residue substitutions, deletions or insertions numbering 0-2 compared to the sequence:
(SEQ ID NO: 1)
GLTYGSPSEGFTWSDGSPVSYENWAYGEPNNYQNVEYCGELKGDPTMSW
NDINCEHLNNWICQ,
wherein the polypeptide or peptidomimetic is immobilized on a carrier,
wherein the sequence comprises YEN residues aligning with the bolded sequence at positions 21-23 of SEQ ID NO: 1; and
wherein the polypeptide or peptidomimetic comprises no more than 100 residues in total.

37-38. (canceled)

39. The composition according to claim 36, wherein the carrier is an inorganic or polymeric nanoparticle.

40-47. (canceled)
